(19) United States
(12) Patent Application Publication
(10) Pub. No.: US 2012/0252756 A1
(43) Pub. Date: Oct. 4, 2012

(54) Pharmaceutical Compositions and Methods for Treating, Controlling, Ameliorating, or Reversing Conditions of the Eye

(76) Inventors: Martin J. Coffey, Pittsford, NY (US); Reza Haque, Randolph, NJ (US); Ngumah Quintus, Scotch Plains, NJ (US); Mohannad Shawer, Webster, NY (US); Jinzhong Zhang, Pittsford, NY (US)

(21) Appl. No.: 13/469,853

(22) Filed: May 11, 2012

Related U.S. Application Data
(63) Continuation-in-part of application No. 13/116,100, filed on May 26, 2011.
(60) Provisional application No. 61/358,463, filed on Jun. 25, 2010.

(51) Int. Cl. A61K 31/717; A61P 27/04; A61P 27/02

(52) U.S. Cl. 514/57

(57) ABSTRACT

A pharmaceutical composition comprises a polyethylene glycol having a molecular weight in the range from about 1,000 to about 10,000, and a water-soluble cellulose derivative having a molecular weight in the range from about 50,000 to 120,000. The composition can further comprise boric acid and/or phosphate, a non-ionic surfactant, and/or an ophthalmic therapeutic agent. The composition is effective in treating, controlling, ameliorating, or reversing one or more conditions or symptoms of dry eye.

### CROSS REFERENCE

This application is a continuation-in-part application, and claims the benefit, of patent application Ser. No. 13/116,100 filed on May 26, 2011, which claims the benefit of Provisional Patent Application No. 61/358,463 filed on Jun. 25, 2010. Both of these applications are incorporated by reference herein.

### BACKGROUND

The present invention relates to compositions and methods for providing comfort to an eye. In particular, the present invention relates to compositions and methods for treating, controlling, ameliorating, or reversing ocular conditions or symptoms of a patient suffering from the condition of dry eye.

Dry eye, or keratoconjunctivitis sicca (“KCS”), often generates the majority of complaints from ophthalmic patients. Unaddressed conditions of dry eye can lead to erosion and abrasion of the epithelial cell surface of the cornea, raising susceptibility to infection. Progression of the disease can lead to ulceration of the cornea, even loss of sight.

A variety of irritants, injuries, and medical conditions predispose individuals to initiation of events that eventually lead to deficiency of the tear film protecting and nourishing the surface of the eye. There are environmental factors such as high altitudes, arid and windy climates, air pollution, desiccating air from central heat and central air conditioning, and exposure to cigarette smoke which can establish and/or enhance deterioration of the quantity and quality of tear production. Even extensive computer use can be a contributing factor as studies have shown significantly decreased blinking rates for users concentrating their attention on computer screens. Some advances in eye care, starting with the introduction of contact lenses, and currently, the popularity of the LASIK procedure for vision correction, have been linked to the recent growth of subject numbers with dry eye. Use of contact lenses can result in absorption of tear film by the lens, with resultant physical irritation of the conjunctiva in the eyelids. LASIK can have a secondary effect of eye injury as nerves often can be severed or ablated during laser refractive surgery, which can lead to at least temporary dry eye syndrome of several months duration.

Some diseases and some physical conditions also can predispose individuals to dry eye disorder. These diseases or conditions include allergies, diabetes, lupus, Parkinson's disease, Sjogren's syndrome, rheumatoid arthritis, rosacea, and others. Medications for other diseases, including diuretics, antidepressants, allergy medications, birth control pills, decongestants and others, may cause or exacerbate dry eye disorders.

Age related changes may induce or exacerbate dry eye as well. Post-menopausal women experience changes in hormonal levels that can instigate or worsen dry eye, and thyroid imbalances may cause similar changes. Finally, aging itself can cause a reduction in lipid production with resultant dry eye.

In the human eye, the tear film covering the ocular surfaces is composed of three layers, from the outermost to the inner most: a lipid layer, an aqueous layer, and a mucous layer. The mucous layer in contact with the ocular surface comprises mucins, which are high-molecular-weight glycoproteins, serving to coat the cornea and provide lubrication thereto. Mucins are secreted by goblet cells residing in the conjunctiva. The middle aqueous layer, which comprises the bulk of the tear film and promotes spreading of the tear film, controlling of infectious agents, and regulating the osmolality, is produced by the lacrimal glands situated in the upper, outer portion of each orbit. The outermost layer is a thin (less than 250 nm) layer comprised of many lipids known as “meibum” or “sebum.” Meibum is secreted by the meibomian glands, located within both the upper and lower eye lids, to form the lipid layer of the tear film, which serves to slow down evaporation of the aqueous layer. Impairment of the production of materials essential to form any of these layers leads to deficiency in the tear film, and eventually the dry eye condition.

Until recently, therapeutic interventions were limited to palliative measures to increase the moisture level of the eye. This is most frequently achieved with instillation of fluids which act as artificial tears, which are in the form of solutions, gels, or ointments. However, these interventions at best provide only short-term relief for the symptoms of dry eye, and none targets the root causes of this disorder to promote the reestablishment of the natural tear film.

Therefore, it is very desirable to develop advantageous compositions and methods, which can effectively promote the natural production and reestablishment of the tear film or ameliorate the impaired ocular surface in dry eye patients. It is also desirable to achieve these compositions and methods with minimal side effects.

### SUMMARY

In general, the present invention provides improved pharmaceutical compositions and methods that can effectively promote the natural production and reestablishment of the tear film or ameliorate the impaired ocular surface in dry eye patients.

In one aspect, the present invention provides these compositions and methods with minimal side effects.

In another aspect, the present invention provides pharmaceutical compositions that comprises one or more compounds that promotes the production of one or more components of the tear film or the repair or amelioration of the impaired ocular surface in dry eye patients.

In still another aspect, the present invention provides a pharmaceutical composition that comprises a polyethylene glycol having a molecular weight in the range from 1,000 to 10,000 Da and a water-soluble cellulose derivative having a molecular weight in the range from 50,000 to 120,000 Da. In one embodiment, such cellulose derivative is a non-ionic water-soluble cellulose derivative.

In yet another aspect, the present invention provides an aqueous pharmaceutical composition that comprises a polyethylene glycol having a molecular weight in the range from 1,000 to 10,000 Da and a water-soluble cellulose derivative having a molecular weight in the range from 50,000 to 120,000 Da. In one embodiment, such a composition is an aqueous solution.

In a further aspect, the present invention provides an aqueous pharmaceutical composition that comprises a polyethylene glycol having a molecular weight in the range from 2000 to 8,000 Da and a non-ionic, water-soluble cellulose derivative having a molecular weight in the range from 60,000 to 100,000 Da. In one embodiment, such a composition is an aqueous solution.

In yet another aspect, the present invention provides a method for treating, controlling, ameliorating, or reversing conditions of dry eye. The method comprises administering to an affected eye a pharmaceutical composition that comprises a polyethylene glycol having a molecular weight in the range from 1,000 to 10,000 Da and a water-soluble cellulose derivative having a molecular weight in the range from 50,000 to 120,000 Da, in an amount and a frequency effective to treat, control, ameliorate, or reverse a condition of dry eye. In one embodiment, such a composition is an aqueous solution.

In still another aspect, the present invention provides a method for treating, controlling, ameliorating, or reversing conditions of dry eye. The method comprises administering to an affected eye a pharmaceutical composition that comprises a polyethylene glycol having a molecular weight in the range from 1,000 to 10,000 Da and a water-soluble cellulose derivative having a molecular weight in the range from 50,000 to 120,000 Da, in an amount and a frequency such that said administering promotes wound healing, improves the protective capacity of the affected cornea, or increases the production of mucins in the affected eye. In one embodiment, such a composition is an aqueous solution.

Other features and advantages of the present invention will become apparent from the following detailed description and claims.

### BRIEF DESCRIPTION OF THE DRAWINGS

FIG. 1 shows the effect of a composition of the present invention on corneal re-epithelization in Riken transformed human corneal epithelial cells. A single horizontal scratch was made to the HCEpiC monolayer. Cells were then incubated with PEG 3350 or HPMC 2910 in basal culture medium. Wound healing was calculated as the difference in the gap width between baseline measurements and after 16-hour incubation and is expressed as percentage gap closure. Bars represent the difference in the gap width between baseline measurements and after 16-hour incubation with treatment groups (n=3); lines on bars represent the standard error of the means. Raw data were analyzed with a one-way ANOVA followed by the Dunnett's Method test. * denotes statistical significance versus vehicle control; p<0.05.

FIG. 2 shows the effect of PEG 3350 and hypromellose 2910 on desiccation-induced HCEpiC cell death. Cells were cultured in complete (HCGS containing) medium until confluent. Cells were pretreated with PEG3350 or HPMC 2910 in basal media for 10 min, followed by desiccation for 0-45 min. Live cells were labeled with calcein (upper panels) and dead cells were labeled with ethidium homodimer (lower panels) using a LIVE/DEAD assay kit (Invitrogen). N=8, *vs. media control at the same time point; p<0.05.

FIG. 3 shows the effect of NaCl hyperosmolarity and PEG 3350 on Riken cell monolayer integrated resistance.

FIG. 4 shows the effect of NaCl hyperosmolarity on normalized resistance of Riken cell monolayer over a 3-hour time course.

FIG. 5 shows the effect of NaCl hyperosmolarity on raw resistance of Riken cell monolayer over a 3-hour time course.

FIG. 6 shows the effect of sucrose hyperosmolarity and PEG 3350 on Riken cell monolayer integrated resistance.

FIG. 7 shows the effect of sucrose hyperosmolarity on Riken cell monolayer normalized resistance over a 24-hour time course.

FIG. 8 shows the effect of sucrose hyperosmolarity on raw resistance of Riken cell monolayer over a 24-hour time course.

FIG. 9 shows the effect of PEG-3350 on HCEpiC MUC1 and MUC16 mRNA levels. Cells were cultured in complete (HCGS containing) medium until confluent. Cells were treated with 3% or 10% PEG-3350 for 4, 8, 18, or 24 hours; or in 10% PEG-3350 for 2 hour followed by 2, 6, 16, or 22 hours. Total RNA was extracted from the cells and QPCR was performed using Taqman MUC 1 or MUC 16 primer/probe sets. (A) MUC1 mRNA; (B) MUC16. N=3, * denoting versus control at the same time point; p<0.05.

FIG. 10 shows the effect of 10% PEG-3350 on pAkt, pERK, pEGFR, and pPI3K activation as shown by western blot. Human corneal epithelial cells (HCEpiC) were treated with 10% PEG-3350 in serum-free media over the course of 16 hours in an attempt to understand the molecular mechanisms behind the observed positive effect on corneal re-epithelization. Cell lysates were collected and assessed for protein activation by western blot using antibodies targeting key phosphorylation sites.

FIG. 11 is a graphical representation of peak phosphorylation time points for pAkt, pERK, and pEGFR. Human corneal epithelial cells (HCEpiC) were treated with 10% PEG-3350 in serum-free media over the course of 16 hours in an attempt to understand the molecular mechanisms behind the observed positive effect on corneal re-epithelization. Cell lysates were collected and assessed for protein activation by western blot using antibodies targeting key phosphorylation sites. Graphs representative of peak protein phosphorylation time-points for respective proteins. Ratio of phosphorylated protein to total, non-phosphorylated was quantified by densitometry.

FIG. 12 shows the distribution of ZO-1 and actin in RT-HCEpiC without 2-hour PEG-3350 pretreatment and after 2-hour incubation with basal or hyperosmotic medium.

FIG. 13 shows the distribution of ZO-1 and actin in RT-HCEpiC with 2-hour 3% PEG-3350 pretreatment and after 2-hour incubation with basal or hyperosmotic medium.

FIG. 14 shows the distribution of ZO-1 and actin in RT-HCEpiC with 2-hour 10% PEG-3350 pretreatment and after 2-hour incubation with basal or hyperosmotic medium.

FIG. 15 shows the comparison of ZO-1 and actin in RT-HCEpiC with or without 2-hour 10% PEG-3350 pretreatment and after 2-hour incubation with basal medium.

FIG. 16 shows the comparison of ZO-1 and actin in RT-HCEpiC without 2-hour 10% PEG-3350 pretreatment and after 2-hour incubation with hyperosmotic medium.

FIG. 17 shows the comparison of ZO-1 and actin in RT-HCEpiC with 2-hour 10% PEG-3350 pretreatment and after 2-hour incubation with hyperosmotic medium.

FIG. 18 shows the effect of sucrose hyperosmolarity and 3% PEG-3350 on Riken cell monolayer integrated resistance.

FIG. 19 shows the effect of sucrose hyperosmolarity and 3% PEG 3350 on normalized resistance of Riken cell monolayer over a 24-hour time course.

FIG. 20 shows the effect of sucrose hyperosmolarity and 3% PEG 3350 on raw resistance of Riken cell monolayer over a 24-hour time course.

### DETAILED DESCRIPTION

Throughout this disclosure, when a numerical range is recited, it should be understood that such a range includes the numerical values of the lower and upper ends.

In general, the present invention provides improved pharmaceutical compositions and methods that can effectively promote the natural production and reestablishment of the tear film or ameliorate the impaired ocular surface in dry eye patients.

In one aspect, the present invention provides these compositions and methods with minimal side effects.

In another aspect, the present invention provides pharmaceutical compositions that comprises one or more compounds that promotes the production of one or more components of the tear film or the repair or amelioration of the impaired ocular surface in dry eye patients.

In still another aspect, the present invention provides a pharmaceutical composition that comprises a polyethylene glycol having a molecular weight in the range from 1,000 to 10,000 Da and a water-soluble cellulose derivative having a molecular weight in the range from 50,000 to 120,000 Da.

In yet another aspect, the present invention provides an aqueous pharmaceutical composition that comprises a polyethylene glycol having a molecular weight in the range from 1,000 to 10,000 Da and a water-soluble cellulose derivative having a molecular weight in the range from 50,000 to 120,000 Da. In one embodiment, such a composition is an aqueous solution.

In a further aspect, the present invention provides an aqueous pharmaceutical composition that comprises a polyethylene glycol having a molecular weight in the range from 2000 to 8,000 Da and a non-ionic, water-soluble cellulose derivative having a molecular weight in the range from 60,000 to 100,000 Da. In one embodiment, such a composition is an aqueous solution.

In yet another aspect, the present invention provides a method for treating, controlling, ameliorating, or reversing one or more conditions of dry eye. The method comprises administering to an affected eye a pharmaceutical composition that comprises a polyethylene glycol having a molecular weight in the range from 1,000 to 10,000 Da and a water-soluble cellulose derivative having a molecular weight in the range from 50,000 to 120,000 Da, in an amount at a frequency effective to treat, control, ameliorate, or reverse a condition of dry eye. In one embodiment, such a condition includes discomfort in the ocular surface, such as a feeling of dryness, grittiness, stinging, or deficiency in aqueous layer, lipid, or mucin production. In another embodiment, such a composition is an aqueous solution.

In still another aspect, the present invention provides a method for treating, controlling, ameliorating, or reversing one or more conditions of dry eye. The method comprises administering to an affected eye a pharmaceutical composition that comprises a polyethylene glycol having a molecular weight in the range from 1,000 to 10,000 Da and a water-soluble cellulose derivative having a molecular weight in the range from 50,000 to 120,000 Da; wherein said administering promotes wound healing, improves the protective capacity of the affected cornea, or increases the production of mucins in the affected eye. In one embodiment, such a composition is an aqueous solution.

In one embodiment, any one of the pharmaceutical compositions of the present invention herein disclosed further comprises one or more ophthalmically acceptable ingredients that can provide benefits to the patients, such as buffers, anti-oxidants, vitamins, viscosity-adjusting materials, tonicity-adjusting materials, preservatives, demulcents, surfactants, pH-adjusting material, etc.

In another embodiment, a pharmaceutical composition of the present invention comprises: (a) a polyethylene glycol having a molecular weight in the range from 1,000 to 10,000 Da; (b) a water-soluble cellulose derivative having a molecular weight in the range from 50,000 to 120,000 Da; and (c) a buffer. In one embodiment, such buffer comprises boric acid and/or phosphate buffer.

In still another embodiment, a pharmaceutical composition of the present invention comprises: (a) a polyethylene glycol having a molecular weight in the range from 1,000 to 10,000 Da; (b) a water-soluble cellulose derivative having a molecular weight in the range from 50,000 to 120,000 Da; (c) a buffer selected from the group consisting of boric acid, phosphate buffer, and mixtures thereof; and (d) a pharmaceutically acceptable preservative.

In yet another embodiment, a pharmaceutical composition of the present invention comprises: (a) a polyethylene glycol having a molecular weight in the range from 1,000 to 10,000 Da; (b) a water-soluble cellulose derivative having a molecular weight in the range from 50,000 to 120,000 Da; and (c) a buffer selected from the group consisting of boric acid, phosphate buffer, and mixtures thereof; (d) a pharmaceutically acceptable preservative; and (e) a preservative efficacy-enhancing material selected from the group consisting of D-glucose, sucrose, maltose, D-mannose, trehalose, glutamic acid, mixtures thereof, wherein said preservative efficacy-enhancing material provides to said pharmaceutical composition an enhanced preservative efficacy against a spore-forming microorganism compared to a composition without said preservative efficacy-enhancing material.

In still another aspect, the polyethylene glycol included in any one of the compositions of the present invention herein disclosed is selected from the group consisting of polyethylene glycols having a molecular weight in the range from about 1,000 to about 10,000 Da. Alternatively, the polyethylene glycol is selected from the group consisting of polyethylene glycols having a molecular weight in the range from about 2,000 to about 10,000 Da; or from about 3,000 to about 8,000 Da. Non-limiting examples of such polyethylene glycol are known under the common names of PEG-1000, PEG-2000, PEG-3350, PEG-4000, PEG-6000, PEG-8000, and PEG-1000. Suitable polyethylene glycols having molecular weight in this range are known under the CTFA (Cosmetic, Toiletry and Fragrance Association) nomenclature as PEG-20, PEG-32, PEG-75, PEG-100, and PEG-150 with molecular weight of 1000, 1450, 3350, 4500, and 8000 Da, respectively. Particularly suitable polyethylene glycols are those having molecular weight in the range from about 2,000 to about 8,000 Da.

The amount of the polyethylene glycol in a composition of the present invention is in the range from about 2 to about 25 percent by weight. Alternatively, the amount of polyethylene glycol in a composition of the present invention is in the range from about 2 to about 20 percent, or from about 3 to about 20 percent, or from about 3 to about 15 percent, or from about 3 to about 12 percent, or from about 3 to about 10 percent, or from about 5 to about 15 percent, or from about 5 to about 12 percent, from about 5 to about 10 percent, or from about 7 to about 25 percent, or from about 7 to about 15 percent, or from about 7 to about 12 percent, or from about 7 to about 10 percent, by weight. In one aspect, the amount of the polymer included in a composition varies in inverse relationship with its molecular weight.

In yet another aspect, the water-soluble cellulose derivative included in any one of the compositions of the present invention herein disclosed is selected from the group consisting of hydroxypropylmethyl cellulose (HPMC), hydroxypropyl cellulose (HPC), hydroxyethyl cellulose (HEC), methyl cellulose, carboxymethyl cellulose (CMC), hydroxypropyl guar, and mixture thereof. In one preferred embodiment, the water-soluble cellulose derivative included in any one of the compositions of the present invention herein disclosed is HPMC, Various HPMC grades having different viscosities are commercially available, for example, from the Dow Chemical Company. Typically, the viscosities of these cellulose derivatives are specified as apparent viscosities of a 2% (by weight) aqueous solution at 20° C. Commercial cellulose derivatives have such apparent viscosity in the range from about 80 to about 1.4,000 cp.

The amount of a water-soluble cellulose derivative in a composition of the present invention is in the range from about 0.1 to about 10 percent by weight. Alternatively, the amount of a water-soluble cellulose derivative in a composition of the present invention is in the range from about 0.1 to about 7 percent, or from about 0.1 to about 5 percent, or from about 0.1 to about 3 percent, or from about 0.1 to about 2 percent, or from about 0.1 to about 1 percent, or from about 0.3 to about 3 percent, from about 0.3 to about 2 percent, or from about 0.3 to about 1 percent, or from about 0.4 to about 1 percent, or from about 0.5 to about 1 percent, or from about 1 to about 3 percent, or from about 1 to about 4 percent, or from about 1 to about 5 percent, by weight.

In still another embodiment, an aqueous pharmaceutical composition of the present invention comprises: (a) a polyethylene glycol having a molecular weight in the range from 1,000 to 10,000 Da at a concentration from about 2 to about 25 percent by weight of the total composition; (b) a water-soluble cellulose derivative having a molecular weight in the range from 50,000 to 120,000 Da at a concentration from about 0.1 to about 10 percent by weight of the total composition; and (c) a buffer selected from the group consisting of boric acid, phosphate buffer, and mixtures thereof.

In a further embodiment, any one of the pharmaceutical compositions of the present invention further comprises a pharmaceutical active ingredient.

Any one of the pharmaceutical compositions or formulations of the present invention herein disclosed can comprise: (a) a polyethylene glycol having a molecular weight in the range from 1,000 to 10,000 Da; (b) a water-soluble cellulose derivative having a molecular weight in the range from 50,000 to 120,000 Da; and (c) a buffer selected from the group consisting of boric acid, phosphate buffer, and mixtures thereof; (d) a pharmaceutically acceptable preservative; and (e) an anti-oxidant. In one embodiment, said anti-oxidant is selected from the group consisting of BHT (butylated hydroxytoluene), thiosulfate salt (such as sodium, potassium, calcium, or magnesium salt), and mixtures thereof.

An embodiment of the pharmaceutical compositions or formulations of the present invention herein disclosed can comprise, consist of, or consists essentially of: (a) a polyethylene glycol having a molecular weight in the range from 1,000 to 10,000 Da at a concentration from about 2 to about 25 percent by weight of the total composition; (b) a water-soluble cellulose derivative having a molecular weight in the range from 50,000 to 120,000 Da at a concentration from about 0.1 to about 10 percent by weight of the total composition; and (c) a buffer selected from the group consisting of boric acid, phosphate buffer, and mixtures thereof; (d) a pharmaceutically acceptable preservative; (e) an anti-oxidant; and (f) water. In one embodiment, said anti-oxidant is selected from the group consisting of BHT (butylated hydroxytoluene), thiosulfate salt (such as sodium, potassium, calcium, or magnesium salt), and mixtures thereof.

A pharmaceutical composition or formulation of the present invention herein disclosed can comprise, consists of, or consists essentially of: (a) a polyethylene glycol having a molecular weight in the range from 1,000 to 10,000 Da; (b) a water-soluble cellulose derivative having a molecular weight in the range from 50,000 to 120,000 Da; and (c) a buffer selected from the group consisting of boric acid, phosphate buffer, and mixtures thereof; (d) a pharmaceutically acceptable preservative; (e) an anti-oxidant selected from the group consisting of BHT, thiosulfate salt (such as sodium, potassium, calcium, or magnesium salt), and mixtures thereof; (f) a preservative efficacy-enhancing material selected from the group consisting of D-glucose, sucrose, maltose, D-mannose, trehalose, glutamic acid, mixtures thereof; and (g) water; wherein the pharmaceutical composition has an enhanced preservative efficacy against a spore-forming microorganism.

In one aspect the preservative included in any one of the pharmaceutical compositions or formulations of the present invention herein disclosed comprises, consists of, or consists essentially of one or more pharmaceutically acceptable alcohols, amines and ammonium-containing compounds, hydrogen peroxide and compounds that produce hydrogen peroxide in said composition (such as carbamide peroxide, carbamide perhydrate, percarbamide, or perborate salts), oxychloro compounds such as chlorine dioxide, zinc compounds, or a mixture thereof. In one embodiment, the pharmaceutically acceptable preservative is selected from the group consisting of polyquaternium-1, -2, -4, -5, -6, -7, -8, -9, -45, -54, -71, and -72. The chemical formulae of these compounds are known in pharmaceutical books.

In one preferred embodiment, the pharmaceutically acceptable preservative is polyquaternium-1, which has the following formula.

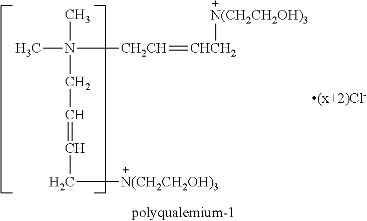

In another embodiment, the pharmaceutically acceptable preservative is selected from the group consisting of a source of hydrogen peroxide (such as perborate, peracetate, or urea peroxide), hydrogen peroxide, stabilized oxychloro complex, and mixtures thereof.

In one aspect, when a composition of the present invention includes a preservative-enhancing material, as disclosed hereinabove, such material provides the composition with an enhanced preservative efficacy against spore-forming microorganisms, otherwise not achievable with a low concentration of a preservative in range that renders the composition comfortable to the user. In one embodiment, such a spore-forming microorganism is a mold or yeast. In another embodiment, such preservative efficacy is that required to meet the European Pharmacopoeia A (“EP-A”) criteria.

In still another embodiment, when a composition of the present invention includes a preservative and a preservative-enhancing material, as disclosed hereinabove, such preservative-enhancing material provides the composition with an enhanced preservative efficacy against spore-forming microorganisms, wherein the preservative is at a concentration that alone does not allow the composition to satisfy the EP-A preservative efficacy criteria.

In another aspect, the spore-forming microorganism is a spore-forming A. brasiliensis.

Procedure for evaluating the preservative efficacy (“PE”) of a pharmaceutical formulation of the present invention against microorganisms

The microorganisms against which the PE of a pharmaceutical formulation of the present invention is evaluated are S. aureus, E. coli, P. aeruginosa, C. albicans, and A. brasiliensis. This procedure applies to the US FDA premarket notification (510(k)) guidance document and USP/ISO/DIS 14730 standard preservative efficacy testing with a 14-day rechallenge. The evaluations were conducted with 3 separate lots of each test solution for each microorganism. Each lot was tested with a different preparation of each microorganism.

Bacterial cells were grown on Tryptic Soy Agar (“TSA”) slants at a temperature in the range from 30 to 35° C. in an incubator for a time period from 18 to 24 hours. Fungal cells were grown on Sabouraud Dextrose Agar (“SDA”) slants at a temperature in the range from 20° C. to 25° C. in an incubator for a time period of 2 to 7 days. Cells were harvested in saline solution (5-10 ml, USP, 0.9% saline, with or without 0.1% Tween 80 surfactant, which was added to each agar slant, followed by gentle agitation with a sterile cotton swab. The cell suspensions were aseptically dispensed into separate sterile polypropylene centrifuge tubes. Cells were harvested by centrifugation at 3000 rpm for 10 minutes, washed one time, and suspended in Saline TS to a concentration of 2×10⁸ cells per ml.

The cell suspension (0.1 ml) was diluted with 20 ml of the test solution to reach a final concentration of from 1.0×10⁻⁵ to 1.0×10⁶ colony-forming units (“CFU”). Phosphate Buffered Saline (“PBS”) was used as a control solution. The inoculated test and control solutions were incubated at a temperature ranging from 20° C. to 25° C. in static culture. At time zero, 1 ml of PBS (USP, pH 7.2) from the control solution was diluted with 9 ml of PBS and serially diluted cells were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

Similarly, at days 7 and 14, a one-milliliter volume from a test solution was added into 9 ml of Dey-Engley neutralizing broth (“DEB”) and serially diluted in DEB and plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20° C. to 25° C. for a period ranging from 2 to 7 days. Developing colonies were counted.

Immediately following the day 14 sampling, test solutions were re-inoculated to give final concentrations of from 1.0×10⁴ to 1.0×10⁵ of each microorganism. At time zero, 1 ml from the inoculum control was added to 9 ml of PBS and subsequent serial dilutions were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

At days 21 and 28, 1 ml from the test articles was added to 9 ml of DEE and again, serial dilutions were plated in triplicate on TSA. Plates were incubated at a temperature ranging from 30 to 35° C. for a period ranging from 2 days to 4 days and developing colonies counted.

Based on the acceptance criteria for bacteria for US Pharmacopeia (“USP”), a solution is acceptable if the concentration of viable bacteria, recovered per milliliter, is reduced by at least 1 log (log to the base 10 or log₁₀) at day 7, by at least 3 logs at day 14, and after a rechallenge at day 14, the concentration of bacteria is reduced by at least 3 logs by day 28. In addition, the solution is acceptable if the concentration of viable yeasts and molds, recovered per milliliter of the solution, remains at or below the initial concentration (within an experimental uncertainty of ±0.5 log) at day 14, and after a rechallenge at day 14, the concentration of viable yeasts and molds remains at or below the initial concentration (within an experimental uncertainty of ±0.5 log) at day 28.

It is notable that the acceptance criteria for a product marketed in Europe are more stringent than those stated above. A pharmaceutical composition meeting such more stringent criteria may be termed “having enhanced preservative efficacy against micro organisms.”

Based on a set of more stringent target acceptance criteria (“EP-A” or European target criteria) for bacteria, a solution is acceptable if the concentration of viable bacteria, recovered per milliliter, is reduced by at least 2 logs (log₁₀) at the end of 6 hours, at least 3 logs at the end of 24 hours, and after a rechallenge at day 14, no bacteria are recovered concentration (“no recovery,” considered to be equal to or greater than 4 logs reduction) by day 28. In addition, the solution is acceptable if the concentration of viable yeasts and molds, recovered per milliliter of the solution, is reduced by at least 2 logs by day 7, and after a rechallenge at day 14, the concentration of viable yeasts and molds remains at or below the initial concentration (within an experimental uncertainty of ±0.5 log) at day 28.

Based on an alternative set of more stringent acceptance criteria (“EP-B” or European acceptable criteria) for bacteria, a solution is acceptable if the concentration of viable bacteria, recovered per milliliter, is reduced by at least 1 log (log₁₀) at the end of 24 hours, at least 3 logs by day 7, and after a rechallenge at day 14, the concentration of bacteria remains at or below the initial concentration (within an experimental uncertainty of ±0.5 log) by day 28. In addition, the solution is acceptable if the concentration of viable yeasts and molds, recovered per milliliter of the solution, is reduced by at least 1 log by day 14, and after a rechallenge at day 14, the concentration of viable yeasts and molds remains at or below the initial concentration (within an experimental uncertainty of ±0.5 log) at day 28.

The foregoing acceptance criteria are summarized in Table 1.

TABLE 1
Preservative Efficacy Acceptance Criteria
Log₁₀ Reduction

| | Time | | | | |
|---|---|---|---|---|---|
| | 6 hour | 24 hour | 7 day | 14 day | 28 day |
| USP: bacteria | — | — | 1 | 3 | No increase |
| EP-A: bacteria | 2 | 3 | — | — | No recovery |
| EP-B: bacteria | — | 1 | 3 | — | No increase |
| USP: fungi | — | — | No | No | No increase |
| | | | increase | increase | |
| EP-A: fungi | — | — | 2 | — | No increase |
| EP-B: fungi | — | — | — | 1 | No increase |

“—” means “not required”

Any one of the pharmaceutical compositions or formulations of the present invention can be in the form of a solution, a suspension, an emulsion, a dispersion, an ointment, or a cream.

Any one of the pharmaceutical compositions or formulations of the present invention is in the form of, or can comprise, a solution or a suspension.

Any one of the pharmaceutical compositions or formulations can be in the form of, or can comprise, an aqueous solution.

Furthermore, an ophthalmic solution of the present invention can comprise an active pharmaceutical ingredient (or therapeutic agent) such as anti-inflammatory agents, antibiotics, immunosuppressive agents, antiviral agents, antifungal agents, antiprotozoal agents, combinations thereof, or mixtures thereof. Non-limiting examples of anti-inflammatory agents include glucocorticosteroids (e.g., for short-term treatment) and non-steroidal anti-inflammatory drugs (“NSAIs”).

Non-limiting examples of the gluccoorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triarcinolone, triamcinolone acetonide, triamcinolone benetonide, triamcinolone hexacetonide, their physiologically acceptable salts, derivatives thereof, combinations thereof, and mixtures thereof. In one embodiment, the therapeutic agent is selected from the group consisting of difluprednate, loteprednol etabonate, prednisolone, combinations thereof, and mixtures thereof.

Non-limiting examples of the NSAIDs are: aminoarylcarboxylic acid derivatives (e.g., enfenamic acid, etofenamate, flufenamic acid, isonixin, meclofenaric acid, mefenamic acid, niflumic acid, talniflumate, terofenamate, tolfenamic acid), arylacetic acid derivatives (e.g., aceclofenac, acemetacin, alclofenac, amfenac, amrtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiaziric acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., arnpiroxicam, droxicam, isoxicarnm, lornoxicam, piroxicam, tenoxicam), ε-acetamidocaproic acid, S-(5′-adenosyl)-L-mrethionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amnikacin, apramnycin, arbekacin, banmbermycins, butirosin, dibekacin, dihydrostreptomycin, fortimicin(s), gentamicin, isepamicin, kanamycin, micronomicin, neomycin, neomycin undecylenate, netilmicin, paromomycin, ribostamnycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonarn, cephacetrile sodium, cephalexin, cephalogiycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumrequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfarnethoxypyrazine, benzylsulfamide, chloramine-B, chloramine-T, dichloramine T, n²-formylsulfisomidine, n⁴-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachiorpyridazine, sulfachrysoidine, sulfacytine, sulfadiazine, sulfadicramide, sulfadimethoxine, sulfadoxine, sulfaethidofadoxine, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methene, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triacinolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyiroInitrin, siccanin, tubercidin, viridin, allylamines (e.g., butenafine, naftifine, terbinafine), imridazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbanates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

In one aspect, the amount of a therapeutic agent is in the range from 0.001 to percent (or alternatively, from 0.005 to 5, or 0.01 to 2, or 0.01 to 1, or 0.01 to 0.5, or 0.1 to 0.5, or 0.1 to 1, or 0.1 to 2, or 0.5 to 2, or 0.5 to 5 percent) by weight of the pharmaceutical composition.

In one embodiment, the pharmaceutical component comprises a fluoroquinolone having Formula I (a new-generation fluoroquinolone antibacterial agent, disclosed in U.S. Pat. No. 5,447,926, which is incorporated herein by reference).

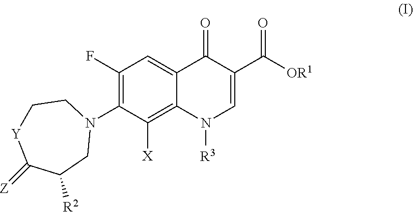

wherein R¹ is selected from the group consisting of hydrogen, unsubstituted C₁-C₅ alkyl groups, substituted C₁-C₅ alkyl groups, C₃-C₇ cycloalkyl groups, unsubstituted C₅-C₂₄ aryl groups, substituted C₅-C₂₄ aryl groups, unsubstituted C₅-C₂ heteroaryl groups, and substituted C₅-C₂₄ heteroaryl groups; R² is selected from the group consisting of hydrogen, unsubstituted amino group, and amino groups substituted with one or two C₁-C₈ alkyl groups; R³ is selected from the group consisting of hydrogen, unsubstituted C₁-C₅ alkyl groups, substituted C₁-C₅ alkyl groups, C₃-C₇ cycloalkyl groups, unsubstituted C₁-C₅ alkoxy groups, substituted C₁-C₅ alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, and substituted C₅-C₂₄ heteroaryloxy groups; X is selected from the group consisting of halogen atoms; Y is selected from the group consisting of CH₂, O, S, SO, SO₂, and NR⁴, wherein R⁴ is selected from the group consisting of hydrogen, unsubstituted C₁-C₅ alkyl groups, substituted C₁-C₅ alkyl groups, and C₃-C₇ cycloalkyl groups; and Z is selected from the group consisting of oxygen and two hydrogen atoms; and wherein when a group is substituted, a substituent is selected from the group consisting of hydroxyl, amino, halogen, C₁-C₅ alkyl, C₁-C₅ alkoxy, C₁-C₅ halogenated alkyl, SO₂, and thiol.

In another embodiment, the pharmaceutical component comprises a fluoroquinolone having Formula I.

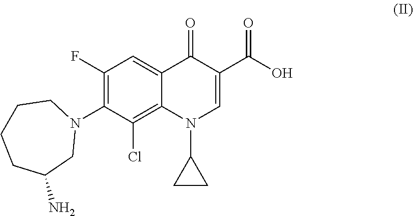

((R)-(+)-7-(3-amino-2,3,4,5,6,7-hexahydro-1H-azepin-1-yl)-8-chloro-1-cyclopropyl-6-fluoro-1,4-dihydro-4-oxoquinoline-3-carboxylic acid).

In still another embodiment, the pharmaceutical component comprises a glucocorticoid receptor agonist having Formulae II or IV, as disclosed in US Patent Application Publication 2006/0116396, which is incorporated herein by reference.

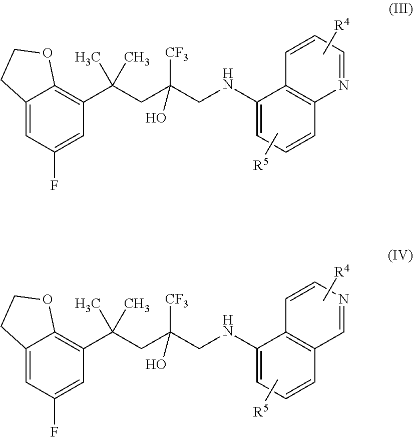

wherein R⁴ and R⁵ are independently selected from the group consisting of hydrogen, halogen, cyano, hydroxy, C₁-C₁₀ (alternatively, C₁-C₅ or C₁-C₃) alkoxy groups, substituted C₁-C₁₀ (alternatively, C₁-C₅ or C₁-C₃) linear or branched alkyl groups, unsubstituted C₃-C₆₀ (alternatively, C₃-C₆ or C₃-C₅) cyclic alkyl groups, and substituted C₃-C₁₀ (alternatively, C₃-C₆ or C₃-C₅) cyclic alkyl groups, wherein when a group is substituted, a substituent is selected from the group consisting of hydroxyl, amino, halogen, C₁-C₅ alkyl, C₁-C₅ alkoxy, C₁-C₅ halogenated alkyl, and thiol.

In yet another embodiment, the pharmaceutical component comprises a glucocorticoid receptor agonist having Formula V (a species of compound having Formula III).

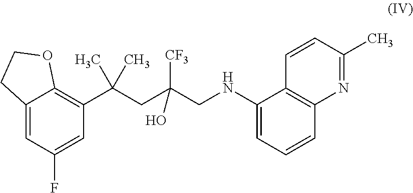

In another embodiment, the therapeutic agent is loteprednol etabonate, an anti-inflammatory agent, having Formula VI.

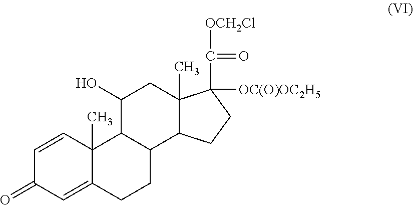

A pharmaceutical composition of the present invention can further comprise a material selected from the group consisting of buffer, tonicity-adjusting agent, viscosity-adjusting agent, pH adjusting agents, antioxidants, chelating agents, and surfactants, and other pharmaceutically acceptable agents, as desired.

An ophthalmic solution of the present invention can be formulated in a physiologically acceptable buffer to regulate pH and tonicity in a range compatible with ophthalmic uses and with any active ingredients present therein. Non-limiting examples of physiologically acceptable buffers include phosphate buffer; a Tris-HCl buffer (comprising tris(hydroxymethyl)aminomethane and HCl); buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris {hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pK₃ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

While the buffer itself is a “tonicity adjusting agent” and a “pH adjusting agent” that broadly maintains the ophthalmic solution at a particular ion concentration and pH, additional “tonicity adjusting agents” can be added to adjust the final tonicity of the solution. Non-limiting examples of tonicity-adjusting agents include, but are not limited to, mannitol, sorbitol, urea, propylene glycol, and glycerin. Also, various salts, including halide salts of a monovalent cation (e.g., NaCl or KCl) can be utilized.

The tonicity adjusting agent, when present, can be in a concentration ranging from about 0.01 to about 10, or from about 0.01 to about 7, or from about 0.01 to about 5, or from about 0.1 to about 2, or from about 0.1 to about 1 percent by weight. In some embodiments where a tonicity adjusting agent is present the solution can contain a single agent or a combination of different tonicity adjusting agents. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg, or from about 290 to 350 mOsm/kg, or from about 240 to 290 mOsm/kg. For certain applications, such as relief of dry eye symptoms or treatment of ocular inflammation, an ophthalmic formulation of the present invention may be desirably hypotonic, such as having tonicity in the range from about 200 to about 270 mOsm/kg, or from about 250 to about 270 mOsm/kg.

Non-limiting examples of viscosity-adjusting agents include synthetic and natural polymers such as poly(acrylic acid) (e.g., the lightly cross-linked poly(acrylic acid) known as Carbopol®, carbomer, or polycarbophil), polysaccharides (e.g., alginic acid, gellan gum, β-glucan, guar gum, gum arabic (a mixture of arabinogalactan ologosaccharides, polysaccharides, and glycoproteins), locust bean gum, pectin, xanthan gum, hyaluronic acid, carboxymethyl starch, carboxymethyl dextran, dextran sulfate, carboxymethyl chitosan, or chondroitin sulfate (e.g., chondroitin sulfate A, chondroitin sulfate B, or chondroitin sulfate C), carrageenan, or curdlan gum), derivatives of cellulose (e.g., carboxymethyl cellulose, methyl cellulose, hydroxypropyl cellulose, hydroxypropyl methyl cellulose, or hydroxyethyl methyl cellulose), or salts thereof. It should be understood that some of the polysaccharides enumerated above may not have naturally occurring salts. Various polyethylene glycols (such as PEG-1000, PEG-3350, PEG-4000, PEG-8000, PEG-10000) may also be considered to be viscosity-adjusting agent.

The amount of a viscosity-adjusting agent may be selected to give the pharmaceutical composition a viscosity in the range from about 2 to about 2,000 centipoises (or mPa·s) (or alternatively, from about 2 to about 1,000, or from about 2 to about 500, or from about 2 to about 100 centipoises), as measured by a Brookfield viscometer (Model RVDV III) at 25° C. and a shear rate of 1-7 sec, with a CPE-40 spindle. The amount of added viscosity-adjusting agent to achieve a certain viscosity can be easily determined experimentally.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnanmic acid, flavonoids, and their salts, esters, and derivatives). In some embodiments, the anti-oxidant comprises ascorbic acid (vitamin C) and its salts and esters; tocopherols (such as α-tocopherol) and tocotrienols (vitamin E), and their salts and esters; BHT; or BHA.

In still another embodiment, the amount of an anti-oxidant in a pharmaceutical formulation of the present invention is in the range from about 0.0001 to about 5 percent by weight of the formulation. Alternatively, the amount of an anti-oxidant is in the range from about 0.001 to about 3 percent, or from about 0.001 to about 1 percent, or from greater than about 0.01 to about 2 percent, or from greater than about 0.01 to about 1 percent, or from greater than about 0.01 to about 0.7 percent, or from greater than about 0.01 to about 0.5 percent, or from greater than about 0.01 to about 0.2 percent, or from greater than about 0.01 to about 0.1 percent, or from greater than about 0.01 to about 0.07 percent, or from greater than about 0.01 to about 0.05 percent, or from greater than about 0.05 to about 0.15 percent, or from greater than about 0.03 to about 0.15 percent by weight of the solution, or from greater than about 0.1 to about 1 percent, or from greater than about 0.1 to about 0.7 percent, or from greater than about 0.1 to about 0.5 percent, or from greater than about 0.1 to about 0.2 percent, or from greater than about 0.1 to about 0.15 percent.

Non-limiting chelating agents include compounds having Formula VII, VIII, or IX.

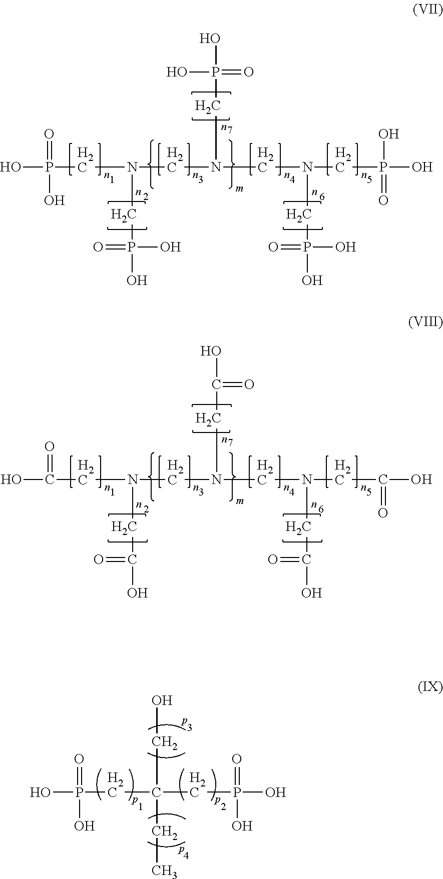

wherein n₁, n₂, n₃, n₄, n₅, n₆, and n₇ are integers independently in the range from 1 to 4, inclusive; m is an integer in the range from 1 to 3, inclusive; p₁, p₂, p₃, and p₄ are independently selected from 0 and integers in the range from 1 to 4, inclusive.

In some embodiments, the chelating agent comprises a compound selected from the group consisting of ethylenediaminetetraacetic acid (“EDTA”), diethylenetriaminepentakis(methylphosphonic acid), etidronic acid, pharmaceutically acceptable salts thereof, and mixtures thereof.

In some other embodiments, the chelating agent comprises tetrasodium salt of etidronic acid (also known as “HAP”, which is available as 30% solution).

In still some other embodiments, the chelating agent comprise EDTA sodium salt.

Ophthalmic solutions of the present invention also can comprise one or more surfactants. Suitable surfactants can include cationic, anionic, non-ionic or amphoteric surfactants. Preferred surfactants are neutral or nonionic surfactants. Non-limiting examples of surfactants suitable for a formulation of the present invention include polyethylene glycol (“PEG,” such as PEG-400, PEG-800, PEG-1000, PEG-3350, PEG-4000, PEG-8000, PEG-10000), polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), poloxamines (synthetic block polymers of ethylene oxide and propylene oxide attached to ethylene diamine, such as those commonly known by their trade names of Tetronic®; e.g., Tetronic® 1508 or Tetronic® 908, etc.), other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In addition to those classes of ingredients disclosed above, a pharmaceutical formulation, such as an ophthalmic solution, of the present invention can further comprise one or more other ingredients, such as vitamins (other than those disclose hereinabove), or other ingredients that provide added health benefits to the users.

A composition of the present invention also can find utility as a contact-lens care. In such a case, it can comprise other known components that are generally used for cleaning and maintenance of contact lenses, as long as these components are compatible with other ingredients in the formulation. In one embodiment, a contact-lens care solution can comprise microabrasives (e.g., polymer microbeads).

In one embodiment, a pharmaceutical composition of the present invention comprises, consists, or consists essentially, of PEG-3350, polysorbate 80, HPMC (hydroxypropylmethylcellulose) 2910 or HPIMC E15LV, boric, a phosphate salt, glycerin, sodium thiosulfate, EDTA salt (such as disodium salt), BHT, polyquaternium-1, and water. HPMC 2910 or HPMC E15LV is available from the Dow Chemical Company.

In one aspect, a pharmaceutical composition of the present invention comprises, consists, or consists essentially, of PEG-3350, polysorbate 80, HPMC (hydroxypropylmethylcellulose) 2910, boric, a phosphate salt, glycerin, sodium thiosulfate, EDTA salt (such as disodium salt), BHT, polyquaternium-1, and water; wherein the composition has a viscosity in the range of 5-30 mPa·s (or cp), and pH in the range of 6-8 (alternatively, from 6.5 to 7.7, or from 6.5 to 7.5, or from 7 to 7.5).

Exemplary concentrations of the components of such a composition are shown in Tables 2 and 3.

TABLE 2

| | | Concentration | Concentration | Concentration |
|---|---|---|---|---|
| | | (wt %, except | (wt %, except | (wt %, except |
| | Present | where | where | where |
| | Invention | indicated) | indicated) | indicated) |
| | Concentration | First | Second | Third |
| | Range | Exemplary | Exemplary | Exemplary |
| Ingredient | (wt %) | Embodiment | Embodiment | Embodiment |
| PEG having molecular | 0.5-20 | 5-15 | 7-12 | 7-12 |
| weight in the range of | | | | |
| 2,000 to 10,000 | | | | |
| (preferably from 2,000 | | | | |
| to, and including, | | | | |
| 8000) | | | | |
| Non-ionic surfactant | 0.1-5 | 0.2-2 | 0.5-1.5 | 0.5-1.5 |
| selected from | | | | |
| Polysorbate 20, 60, | | | | |
| and 80 | | | | |
| Non-ionic, water- | 0.05-3 | 0.1-2 | 0.2-1.5 | 0.2-1.5 |
| soluble cellulose | | | | |
| derivative selected | | | | |
| from HPMC, HEC, | | | | |
| HPC, and methyl | | | | |
| cellulose | | | | |
| Boric acid NF | 0.05-2 | 0.1-1.5 | 0.1-1 | 0.1-1 |
| D-Glucose or sucrose | 0-3 | 0-0.5 | 0 | 0.001-0.5 |
| Polyol (e.g., glycerin, | 0-3 | 0.01-2 | 0.2-1 | 0.2-1 |
| propylene glycol, or | | | | |
| mixtures thereof) | | | | |
| Sodium phosphate | q.s. for | 0.01-0.3 | 0.05-0.2 | 0.05-0.2 |
| dibasic anhydrous | desired buffer | | | |
| | pH | | | |
| Sodium phosphate | q.s. for | 0.005-0.1 | 0.005-0.05 | 0.005-0.05 |
| monobasic | desired buffer | | | |
| monohydrate | pH | | | |
| Sodium thiosulfate | 0.01-0.5 | 0.02-0.3 | 0.03-0.1 | 0.03-0.1 |
| pentahydrate | | | | |
| Antioxidant (e.g., | 0-1 | 0.001-0.5 | 0.01-0.08 | 0.01-0.08 |
| BHT NF) | | | | |
| Ophthalmically | 1-1,000 ppm | 1-500 ppm | 1-300 ppm | 1-300 ppm |
| acceptable | | | | |
| preservative | | | | |
| Chelating agent | 0-1 | 0.001-0.5 | 0.01-0.2 | 0.01-0.2 |
| Water for injection | q.s. 100% | q.s. 100% | q.s. 100% | q.s. 100% |
| USP/EP | | | | |
| pH | 5.5-8 | 6.5-8 | 7.3-7.5 | 7.3-7.5 |
| Osmolality, mOsm/kg | 200-400 | 260-350 | 320-350 | 320-350 |
| Viscosity, cp or mPa · s | 2-2000 | 2-500 | 5-50 | 5-50 |

TABLE 3

| | | Example 1 | |
|---|---|---|---|
| | | Preferred | Example 2 |
| | Present | Concen- | More Preferred |
| | Invention | tration | Concentration |
| | Concentration | (wt %, | (wt %, except |
| | Range | except where | where |
| Ingredient | (wt %) | indicated) | indicated) |
| PEG-3350 NF | 0.5-20 | 5-15 | 10 |
| Polysorbate 80 NF | 0.1-5 | 0.5-2 | 1 |
| HPMC E15LV (USP) | 0.05-3 | 0.1-1 | 0.5 |
| Boric acid NF | 0.05-2 | 0.1-1 | 0.62 |
| D-Glucose or sucrose | 0-3 | 0-0.5 | 0 |
| | | or 0.01-0.5 | |
| Glycerin | 0-3 | 0-1.5 | 0.2 |
| Sodium phosphate | q.s. for desired | 0.01-0.3 | 0.129 |
| dibasic anhydrous | buffer pH | | |
| Sodium phosphate | q.s. for desired | 0.005-0.1 | 0.012 |
| monobasic monohydrate | buffer pH | | |
| Sodium thiosulfate | 0.01-0.5 | 0.02-0.3 | 0.05 |
| pentahydrate | | | |
| BHT NF | 0-1 | 0.001-0.2 | 0.01 |
| Polyquaternium-1 | 1-50 ppm | 1-30 ppm | 10 ppm |
| EDTA disodium | 0-1 | 0.001-0.5 | 0.011 |
| dihydrate | | | |
| Water for injection | q.s. 100% | q.s. 100% | q.s. 100% |
| USP/EP | | | |
| pH | 5.5-8 | 6.5-8 | 7.3-7.5 |
| Osmolality, mOsm/kg | 200-400 | 260-350 | 320-350 |
| Viscosity, cp or mPa · s | 2-2000 | 2-500 | 5-30 |

In another aspect, the present invention provides a method for making an ophthalmic pharmaceutical formulation for treating, controlling, ameliorating, or reversing a condition (such as irritation, discomfort, a feeling of dryness, grittiness, or stinging in the eye, or deficiency in aqueous, lipid, or mucous layer) of a dry eye patient. The method comprises combining: (a) a polyethylene glycol having a molecular weight in the range from 1,000 to 10,000 Da; (b) a water-soluble cellulose derivative having a molecular weight in the range from 50,000 to 120,000 Da; and (c) an ophthalmically acceptable carrier. In one embodiment, such an ophthalmically acceptable carrier comprises water, and such a pharmaceutical formulation is an aqueous solution.

In another aspect, the present invention provides a method for making an ophthalmic pharmaceutical formulation for treating, controlling, ameliorating, or reversing a condition (such as irritation, discomfort, a feeling of dryness, grittiness, or stinging in the eye, or deficiency in aqueous, lipid, or mucous layer) of a dry eye patient. The method comprises combining: (a) a polyethylene glycol having a molecular weight in the range from 1,000 to 10,000 Da at a concentration from about 5 to about 15 percent of the total composition; (b) a water-soluble cellulose derivative having a molecular weight in the range from 50,000 to 120,000 Da at a concentration from about 0.5 to about 2 percent of the total composition; and (c) an ophthalmically acceptable carrier. In one embodiment, such an ophthalmically acceptable carrier comprises water, and such a pharmaceutical formulation is an aqueous solution.

In still another aspect, the present invention provides a method for making an ophthalmic pharmaceutical formulation for treating, controlling, or ameliorating a condition (such as irritation or discomfort in the eye) of a dry eye patient. The method comprises combining the ingredients listed in Tables 2 and 3 at the respective concentrations to produce the ophthalmic formulation.

In yet another aspect, the method further comprises the step of mixing the combined ingredients to achieve substantial uniformity.

In yet another aspect, the method further comprises the steps of sterilizing the formulation to produce a sterilized formulation and packaging the sterilized formulation in suitable containers.

In one embodiment, the method can also comprises: (1) adding and mixing some materials together to produce a first mixture; and (2) adding the remaining materials to the first mixture while mixing continues to produce the composition.

In another embodiment, the method can also comprises: (1) adding and mixing some materials together to produce a first mixture; (2) adding and mixing the remaining materials together to produce a second mixture; and (3) combining the first mixture the second mixture while mixing continues to produce the composition.

Further non-limiting embodiments of the present invention are shown in the following tables.

### Example 3

### Ophthalmic Formulation with NSAID Anti-Inflammatory Drug

The following ingredients are combined to produce such a formulation.

| | % w/w |
|---|---|
| Ingredient | (except otherwise indicated) |
| PEG-3350 NF | 12 |
| Polysorbate 80 NF | 1.5 |
| HPMC E15LV (USP) | 0.4-0.7 |
| Boric acid NF | 0.5 |
| Glycerin | 0.4 |
| Sodium phosphate dibasic anhydrous | 0.14 |
| Sodium phosphate monobasic monohydrate | 0.01 |
| Sodium thiosulfate pentahydrate | 0.06 |
| BHT NF | 0.02 |
| Polyquaternium-1 | 15 ppm |
| EDTA disodium dihydrate | 0.006 |
| Bromfenac | 0.06-0.1 |
| Water for injection USP/EP | q.s. 100% |
| pH | 7.3-7.5 |
| Osmolality, mOsm/kg | 290-330 |
| Viscosity, cp or mPa · s | 5-10 |

### Example 4

### Ophthalmic Formulation for Treating or Controlling High Intraocular Pressure

The following ingredients are combined to produce an exemplary formulation for treating or controlling high intraocular pressure.

| | % w/w |
|---|---|
| Ingredient | (except otherwise indicated) |
| PEG-3350 NF | 10 |
| Polysorbate 80 NF | 1 |
| HPMC E15LV (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Timolol maleate | 0.5 |
| Dorzolamide hydrochloride | 2 |
| Water for injection USP/EP | q.s. 100% |
| pH | 7.3-7.5 |
| Osmolality, mOsm/kg | 290-330 |
| Viscosity, cp or mPa · s | 5-30 |

### Example 5

### Ophthalmic Formulation for Treating or Controlling Eye Infection

The following ingredients are combined to produce such a formulation.

| | % w/w |
|---|---|
| Ingredient | (except otherwise indicated) |
| PEG-4000 NF | 10 |
| Polysorbate 60 NF | 1 |
| HPMC 2910 (USP) | 0.4-0.7 |
| Boric acid NF | 0.5-0.7 |
| Glycerin | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Compound having Formula II | 0.6 |
| Water for injection USP/EP | q.s. 100% |
| pH | 7.3-7.5 |
| Osmolality, mOsm/kg | 290-340 |
| Viscosity, cp or mPa · s | 5-30 |

### Example 6

### Ophthalmic Formulation for Treating or Controlling Eye Infection

The following ingredients are combined to produce such a formulation.

| | % w/w |
|---|---|
| Ingredient | (except otherwise indicated) |
| PEG-4000 NF | 10 |
| Polysorbate 60 NF | 1 |
| HPMC 2910 (USP) | 0.4-0.7 |
| Boric acid NF | 0.5-0.7 |
| Glycerin | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Moxifloxacin | 0.2-0.6 |
| Water for injection USP/EP | q.s. 100% |
| pH | 7.3-7.5 |
| Osmolality, mOsm/kg | 290-340 |
| Viscosity, cp or mPa · s | 5-30 |

### Example 7

### Ophthalmic Formulation for Treating or Controlling Eye Infection

The following ingredients are combined to produce such a formulation.

| | % w/w |
|---|---|
| Ingredient | (except otherwise indicated) |
| PEG-3350 NF | 10 |
| Polysorbate 80 NF | 1 |
| HPMC E15LV (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Compound having Formula II | 0.3-0.8 |
| Water for injection USP/EP | q.s. 100% |
| pH | 7.3-7.5 |
| Osmolality, mOsm/kg | 290-330 |
| Viscosity, cp or mPa · s | 5-30 |

### Example 8

### Ophthalmic Formulation for Treating or Controlling Eye Allergy

The following ingredients are combined to produce an exemplary formulation for treating or controlling eye allergy.

| | % w/w |
|---|---|
| Ingredient | (except otherwise indicated) |
| PEG-3350 NF | 10 |
| Polysorbate 80 NF | 1 |
| HPMC E15LV (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Ketotifen fumarate | 0.02-0.04 |
| Water for injection USP/EP | q.s. 100% |
| pH | 7.3-7.5 |
| Osmolality, mOsm/kg | 260-300 |
| Viscosity, cp or mPa · s | 5-20 |

### Example 9

### Ophthalmic Formulation for Treating or Controlling Eye Allergy

The following ingredients are combined to produce an exemplary formulation for treating or controlling eye allergy.

| | % w/w |
|---|---|
| Ingredient | (except otherwise indicated) |
| PEG-3350 NF | 10 |
| Polysorbate 80 NF | 1 |
| HPMC E15LV (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Olapatadine hydrochloride | 0.1 |
| Water for injection USP/EP | q.s. 100% |
| pH | 7.3-7.5 |
| Osmolality, mOsm/kg | 260-300 |
| Viscosity, cp or mPa · s | 5-20 |

### Example 10

### Ophthalmic Formulation for Treating or Controlling Eye Infection

The following ingredients are combined to produce an exemplary formulation for treating or controlling eye infection.

| | % w/w |
|---|---|
| Ingredient | (except otherwise indicated) |
| PEG-3350 NF | 10 |
| Polysorbate 80 NF | 1 |
| HPMC 2910 (USP) | 0.4-0.7 |
| Carbomer 980 | 0.05-0.15 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| HAP (30%) | 0.05-0.15 |
| Vitamin E TPGS | 0.1 |
| Compound having Formula II | 0.3-0.8 |
| Water for injection USP/EP | q.s. 100% |
| pH | 7.3-7.5 |
| Osmolality, mOsm/kg | 260-300 |
| Viscosity, cp or mPa · s | 5-20 |

### Example 11

### Ophthalmic Formulation for Treating or Controlling Eye Infection

The following ingredients are combined to produce an exemplary formulation for treating or controlling eye infection.

| | % w/w |
|---|---|
| Ingredient | (except otherwise indicated) |
| PEG-4000 NF | 10 |
| Polysorbate 60 NF | 1 |
| HPMC E15LV (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| Benzalkonium chloride | 50 ppm |
| EDTA disodium dihydrate | 0.005-0.01 |
| Compound having Formula II | 0.3-0.6 |
| Water for injection USP/EP | q.s. 100% |
| pH | 7.3-7.5 |
| Osmolality, mOsm/kg | 290-330 |
| Viscosity, cp or mPa · s | 5-30 |

### Example 12

### Ophthalmic Formulation for Treating or Controlling Eye Inflammation

The following ingredients are combined to produce an exemplary formulation for treating or controlling eye inflammation.

| | % w/w |
|---|---|
| Ingredient | (except otherwise indicated) |
| PEG-3350 NF | 10 |
| Polysorbate 80 NF | 1 |
| HPMC 2910 (USP) | 0.4-0.7 |
| Hydroxyethyl cellulose | 0.05-0.15 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| HAP (30%) | 0.05-0.15 |
| Loteprednol etabonate | 0.3-1 |
| Water for injection USP/EP | q.s. 100% |
| pH | 7.3-7.5 |
| Osmolality, mOsm/kg | 260-300 |
| Viscosity, cp or mPa · s | 5-50 |

### Example 13

### Ophthalmic Formulation for Treating or Controlling Eye Inflammation

The following ingredients are combined to produce an exemplary PGP-4 formulation for treating or controlling eye inflammation.

| | % w/w |
|---|---|
| Ingredient | (except otherwise indicated) |
| PEG-3350 NF | 8-12 |
| Polysorbate 80 NF | 0.5-1.5 |
| HPMC 2910 (USP) | 0.4-0.7 |
| Hydroxyethyl cellulose | 0.05-0.15 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| HAP (30%) | 0.05-0.15 |
| Compound having Formula V | 0.3-1 |
| Water for injection USP/EP | q.s. 100% |
| pH | 7.3-7.5 |
| Osmolality, mOsm/kg | 260-300 |
| Viscosity, cp or mPa · s | 5-50 |

### Example 14

### Ophthalmic Formulation for Treating or Controlling Eye Inflammation

The following ingredients are combined to produce an exemplary formulation for treating or controlling eye inflammation. Ingredient % w/w (except otherwise indicated)

| | % w/w |
|---|---|
| Ingredient | (except otherwise indicated) |
| PEG-3350 NF | 10 |
| Polysorbate 80 NF | 1 |
| HPMC 2910 (USP) | 0.4-0.7 |
| Mannitol | 0.5-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| HAP (30%) | 0.05-0.15 |
| Dexamethasone | 0.1 |
| Water for injection USP/EP | q.s. 100% |
| pH | 7.3-7.5 |
| Osmolality, mOsm/kg | 260-300 |
| Viscosity, cp or mPa · s | 5-50 |

### Example 15

### Ophthalmic Formulation for Treating or Controlling Intraocular Pressure

The following ingredients are combined to produce an exemplary formulation for treating or controlling intraocular pressure. The following ingredients are combined to produce an exemplary formulation for treating or controlling eye inflammation.

| | % w/w |
|---|---|
| Ingredient | (except otherwise indicated) |
| PEG-6000 | 5-10 |
| Polysorbate 80 NF | 1-2 |
| HPMC 2910 (USP) | 0.4-0.7 |
| Carboxymethyl cellulose | 0.05-0.15 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| HAP (30%) | 0.05-0.15 |
| Brimonidine tartrate | 0.5 |
| Timolol maleate | 0.7 |
| Water for injection USP/EP | q.s. 100% |
| pH | 7.3-7.5 |
| Osmolality, mOsm/kg | 260-300 |
| Viscosity, cp or mPa · s | 5-50 |

### Example 16

### Formulation Comprising a Second Preservative

The following ingredients are combined to produce an exemplary formulation. This formulation may be used as a vehicle for an ophthalmic active agent or as a contact-lens treating, cleaning, wetting, or storing solution.

The following ingredients are combined to produce an exemplary formulation for treating or controlling eye inflammation.

| | % w/w |
|---|---|
| Ingredient | (except otherwise indicated) |
| PEG-3350 NF | 10 |
| Polysorbate 80 NF | 1 |
| HPMC 2910 (USP) | 0.4-0.7 |
| Boric acid NF | 0.5-0.7 |
| Propylene glycol | 0.4-0.6 |
| Sodium phosphate dibasic anhydrous | 0.1-0.2 |
| Sodium phosphate monobasic monohydrate | 0.01-0.02 |
| Sodium thiosulfate pentahydrate | 0.05-0.07 |
| BHT NF | 0.01-0.03 |
| PQ-1 | 10 ppm |
| Stabilized oxychloro complex | 0.005-0.015 |
| HAP (30%) | 0.05-0.15 |
| Anthocyanin (anti-oxidant) | 0.05 |
| Compound having Formula V | 0.3-1 |
| Water for injection USP/EP | q.s. 100% |
| pH | 7.3-7.5 |
| Osmolality, mOsm/kg | 260-300 |
| Viscosity, cp or mPa · s | 5-50 |

In another aspect, an ophthalmic solution of the present invention, as described in Table 2, can be used to treat, control, or ameliorate conditions or symptoms associated with dry eye, inflammation, or allergy of the eye.

In still another aspect, an ophthalmic solution of the present invention, as described in Table 2, can be used to promote healing of an impaired ocular surface, wherein such impairment is caused by dryness, wounding, or irritation.

In still another aspect, the present invention provides methods of making and using a pharmaceutical formulation of the present invention. Any of the materials, compounds, and ingredients disclosed herein is applicable for use with or inclusion in any method of the present invention.

In one embodiment, the method comprises: (a) combining (i) a pharmaceutically acceptable carrier; (ii) a polyethylene glycol having a molecular weight in the range from 1,000 to 10,000 Da; and (iii) a water-soluble cellulose derivative having a molecular weight in the range from 50,000 to 120,000 Da; and (b) mixing ingredients (i), (ii), and (iii) together for a time sufficient to produce a substantially uniform pharmaceutical composition.

In another embodiment, the method further comprises adding one or more ingredients selected from the group consisting of therapeutic agents, buffers, tonicity adjusting agents, surfactants, viscosity-adjusting agents, and other pharmaceutically acceptable agents to the pharmaceutical composition. The therapeutic agents can be selected from the group of anti-inflammatory agents, agents for lowering intraocular pressure, ocular neuroprotectants, antibiotics, immunosuppressive agents, anti-allergic agents, antiviral agents, antifungal agents, antiprotozoal agents, and mixtures thereof. Non-limiting examples of each of these classes of agents, compounds, and ingredients are disclosed throughout the present specification.

In still another aspect, the pharmaceutically acceptable carrier comprises boric acid and a phosphate buffer.

### Testing 1: Composition of the Present Invention Shows Statistically Significant Improvement Over Baseline for all Primary and Secondary Endpoints of Dry Eye

A randomized, multicenter study lasting 12 weeks and involving 73 patients was conducted to assess the effectiveness of a composition of the present invention in ameliorating the conditions or symptoms of dry eye. The study composition is shown in the following Table T-1-1. Each subject received one drop of the composition twice daily in both eyes.

TABLE T-1-1

| | Concentration |
|---|---|
| Ingredient | ( wt % except otherwise indicated) |
| PEG-3350 NF | 10 |
| Polysorbate 80 NF | 1 |
| HPMC E15LV | 1 |
| Boric acid NF | 0.5 |
| Sucrose NF | 0.5 |
| Glycerin | 0 |
| Sodium phosphate dibasic | 0.142 |
| anhydrous | |
| Sodium phosphate monobasic | 0 |
| monohydrate | |
| Sodium thiosulfate pentahydrate | 0.05 |
| BHT NF | 0 |
| PAPB HCl, 20% solution | 0 |
| Polyquaternium-1 | 4 ppm |
| EDTA dehydrate | 0 |
| pH | 7.6 |
| Osmolality, mOsm/kg | 313 |

### Co-Primary Endpoints

The total corneal staining (“TCS”) score (sum of 5 designated areas in the cornea, at Week 12)
The worst baseline dry eye symptoms (ocular discomfort, dryness, grittiness, and stinging) visual analog scale (“VAS”) score at Week 12 Secondary Efficacy Endpoints
Percent of subjects (study eyes) with complete resolution of central corneal staining
Change from Baseline values for conjunctival staining as determined by the sum of Lissamine green conjunctival staining scores in each of the six conjunctival regions
Percent of subjects who achieve ≧10 mm wetting using the Schirmer tests (without anesthesia)
Change from baseline values in tear fluid secretion in mm as measured by the Schirmer test (unanesthesized)
Percent of subjects (study eye) with complete resolution of total corneal staining

### Safety Endpoints

Proportion of subjects with ocular treatment-emergent adverse events (“TEAEs”): Fifteen of 71 subjects (or 21.1%) experienced at least one TEAEs. However, none of the TEAEs was serious enough to require premature discontinuation. Three patients (or 4.5%) showed an increased intraocular pressure (“IOP”) from baseline value of ≧5 mm Hg, but less than 10 mm Hg. None of the patients showed an increased LOP of ≧10 mm Hg. Thus, the composition judged to be safe for use.

Results of the study at 12 weeks are shown in Table T-1-2.

TABLE T-1-2

| | | | Change from |
|---|---|---|---|
| | | Mean (SD) | Baseline |
| Endpoint | Visit | (N = 73) | (%) |
| Total corneal staining | Baseline | 6.8 (2.9) | |
| | Week 12 | 3.8 (3.3) | −44.1 |
| Worst VAS | Baseline | 6.0 (2.47) | |
| | Week 12 | 2.2 (2.30) | −63.3 |
| Conjunctival staining | Baseline | 5.1 (4.5) | |
| | Week 12 | 3.6 (3.6) | −29.4 |
| Schirmer test | Baseline | 3.8 (2.5) | |
| | Week 12 | 7.4 (8.2) | 94.7 |

### CONCLUSION

The present composition significantly decreased total corneal staining, conjunctival staining, and worst VAS score, and significantly increased tear production (as shown by the Schirmer test) after 12 weeks of BID administration of I drop in the affected eyes. Thus, it was demonstrated that a composition such as the present composition was effective in treating, controlling, ameliorating, or reversing conditions or symptoms of dry eye. In addition, it was also demonstrated that the deficiency in tear production was reversed.

### Testing 2: Compositions of the Present Invention Promotes Corneal Re-Epithelization

### Introduction

Dry eye is a disorder of the ocular surface due to tear deficiency, excessive tear evaporation, or incorrect composition of tears. The resulting desiccation of the ocular surface results in ocular irritation and discomfort.

We have developed an in vitro wound healing model using transformed human corneal epithelial cells insulted with a scratch to the monolayer and monitored for growth into the resulting cell-free gap. Heparin-binding endothelial growth factor (“HB-EGF”) has been shown in the literature to stimulate corneal wound healing using in vivo and in vitro models, and increased HB-EGF expression is observed in the process of corneal wound healing (see; e.g., D. M. Foreman et al., “A Simple Organ Culture Model for Assessing the Effects of Growth Factors on Corneal Re-epithelization,” Exp. Eye Res., Vol. 62, 555-64 (1996); A. Wells, “EGF Receptor,” IJBCB, Vol. 31, 637-43 (1999)). In the scratch assay model system presented here, we have found that HB-EGF has been shown to provide consistent results as a positive assay control in the Riken human corneal epithelial cell line (“RT-HCEpiC”).

The current study determines the ability of PEG 3350 and iPMC 2910 to contribute to corneal re-epithelization following injury to the RT-HCEpiC.

### Methods.

### Monolayer Scratch Assay

A vertical line was drawn on the bottom of the plate at the base of each well of a 24-well plate with a marker, and images were taken for measurement at the intersection of this line and the cell-free gap. RT-HCEpiC were prepared in a suspension of 2.5×105 cells/ml in complete medium, and 500 μl cell suspensions were added to each well. Plates were incubated at 37° C., 5% CO₂, and 95% humidity until a complete monolayer formed. When the cells attained confluence, medium was removed from wells and replaced with basal medium without growth factors. The cells were serum-starved in the incubator for 18 h. After this incubation, 500 μl HBSS was added to each well. The monolayer was artificially disrupted by a single horizontal scratch with a sterile P200 pipette tip. The HBSS was aspirated and wells were washed once more with HBSS. Finally, the treatment solutions in basal medium were applied to the appropriate wells. Baseline images of the monolayer gaps were taken, and cells were returned to the incubator for re-epithelization of the cell gap for 16 h. At this point, cells were examined and photographed to document closure of the monolayer gap using a light microscope.

### Experimental Design and Schedule Summary

TABLE T-2-1

| | | | Day 3 | |
|---|---|---|---|---|
| | | | Monolayer scratch, | |
| | | | applied treatments, | |
| | | | then image for | |
| Group* | Day 1 | Day 2 | baseline data | Day 4 |
| 1 | Seed RT- | Remove | Vehicle Control | Image |
| | HCEpiC | complete | (baseline medium) | cell-free |
| 2 | (2.5 × 10⁵ | medium and | 10 ng/mL HB-EGF | gaps at |
| 3 | cells/ml) in | replace with | HPMC 2910 (1%) | 16 hours |
| 4 | complete | basal medium. | PEG-3350 (1%) | and |
| 5 | DMEM/F12 | Incubate for | PEG-3350 (3%) | determine |
| 6 | medium | 18 hours | PEG-3350 (10%) | gap widths |

*Each treatment assessed in triplicate

### Data Analysis

Cells were examined and photographed after scratch and after 16 hours incubation with treatment to monitor closure of the cell-free gap using a light microscope. The percent closure was determined using the following equation.

((gap width_{baseline}−gap width_{16 h})/gap width_{bsline})×100=percent closure

Comparisons were made between treatment groups and the vehicle controls to determine the effect of the treatment on re-epithelization. Statistical analysis of the net reduction in gap width was conducted with a one-way ANOVA followed by the Dunnett's Method test, and P<0.05 is considered significant. All data was analyzed utilizing the statistical analysis software JMP (SAS Institute, Cary, N.C.).

### Results

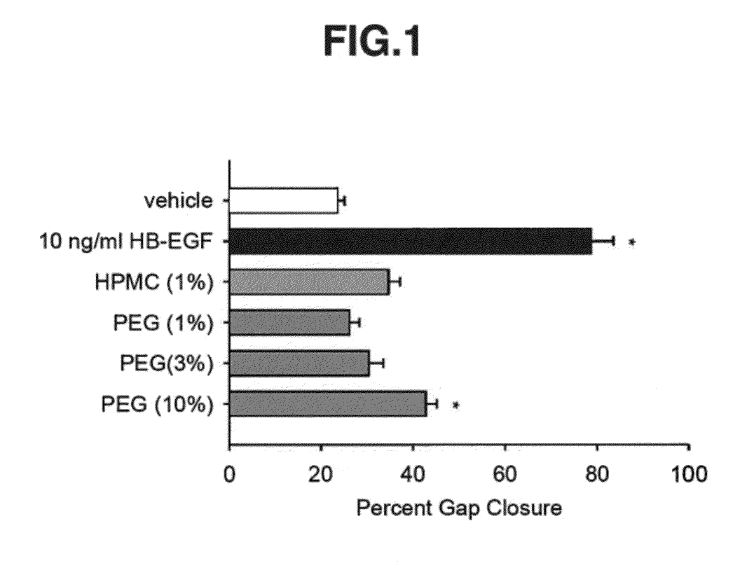

Both 10 ng/mL HB-EGF and PEG 3350 (10%) significantly increased corneal re-epithelization 16 hours after wounding (FIG. 1).

PEG 3350 enhanced wound healing at a concentration of 10%. The effect appeared to be dose-dependent, however no significant effect was observed with 1% or 3% PEG 3350.

There was no effect of HPMC 2910 (1%) on corneal re-epithelization (FIG. 1).

In a preliminary experiment, 0.1% and 0.3% HPMC 2910 were also tested. However, neither a significant effect on corneal re-epithelization nor a dose-dependent response was observed.

### Summary of Findings

PEG 3350 (10%) significantly increased corneal re-epithelization of HCEpiC, while 1% and 3% PEG 3350 and HPMC 2910 (1%) were without effect.

### Testing 3: Composition of the Present Invention Decreases Desiccation-Induced Cell Death in Transformed Human Corneal Epithelial Cells

### Introduction

Several published studies have used an in vitro desiccation model to determine the effect of polymers, lubricating agents and protective agents on the ocular surface (see; e.g., J. L. Ubels et al., “Preclinical Investigation of the Efficacy of an Artificial Tear Solution Containing Hydroxypropyl Guar as a Gelling Agent,” Curr. Eye Res., Vol. 28, 437-44 (2004); T. Matsuo, “Trehalose Protects Corneal Epithelial Cells From Death by Drying,” Br. Ophthalmol., Vol. 85, 610-12 (2001); K. Paulsen et al., “Lubricating Agents Differ in Their Protection of Cultured Human Epithelial Cells Against Desiccation.” Med. Sci. Monit., Vol. 14, P112-16 (2008)). In this model, corneal epithelial cells are exposed to the test agent, then after removal of the test agent, are subjected to drying. Cell viability is then measured. We have established an in vitro desiccation model in transformed human corneal epithelial cells (“HCEpiC”). This study is to determine the ability of a composition of the present invention to protect cells from desiccation-induced cell death.

### Method

### Design

Transformed human corneal epithelial cells from ATCC (T-HCEpiC) were seeded in 4 black-walled 96-well plates at 1.25×104 cells/well in EpiLife medium +1% Human Corneal Growth Supplement (“HCGS”; containing bovine pituiutary extract, bovine insulin, hydrocortisone, bovine transferrin and mouse epidermal growth factor) and cultured until confluent. The medium was removed from the cells and they were pre-treated with basal medium or HPMC 2910 (0.1-1%) or PEG 3350 (1-10%) in basal medium for 10 min (Table T-3-1). Plates were then placed in a tissue culture hood without air-flow for 0, 15, 30 and 45 minutes. Cell viability was assessed using a LIVE/DEAD viability/cytotoxicity kit (Invitrogen).

TABLE T-3-1

| | | Day 2 | |
|---|---|---|---|
| | | Cells were incubated in | |
| | | basal EpiLife for 18 h, | |
| | | pre-treated with PEG 3350 | |
| | | or HPMC 2910 in basal | |
| | | media for 10 min, followed by | |
| | | exposure to desiccation | |
| Group* | Day 1 | for 0-45 minutes. | Day 2 |
| 1 | Seeded T- | Control (basal medium) | After |
| 2 | HCEpiC in 4 96 | PEG-3350 (1%) | exposure to |
| 3 | well plates at | PEG-3350 (3%) | desiccation a |
| 4 | 1.25 × 104 | PE-3350 (10%) | LIVE/DEAD |
| 5 | cells/well | HPMC 2910 (0.1%) | cell viability |
| 6 | | HPMC 2910 (0.3%) | assay was |
| | | HPMC2910 (1%) | performed. |
| 7 | | | |

*denotes eight wells per group.

### Data Analysis

Background fluorescence from the medium alone was subtracted. Changes in levels of live and dead cells were expressed in RFU.

Data were expressed as mean±SEM. Statistical analysis was performed using a two-way ANOVA-Tukey Kramer test (factor I was desiccation time; factor 2 was OTC dry eye drop) using JMP 8 software (SAS Institute, Cary, N.C.). p<0.05 was considered statistically significant. Data were analyzed either directly or after Box-Cox transformations.

### Results

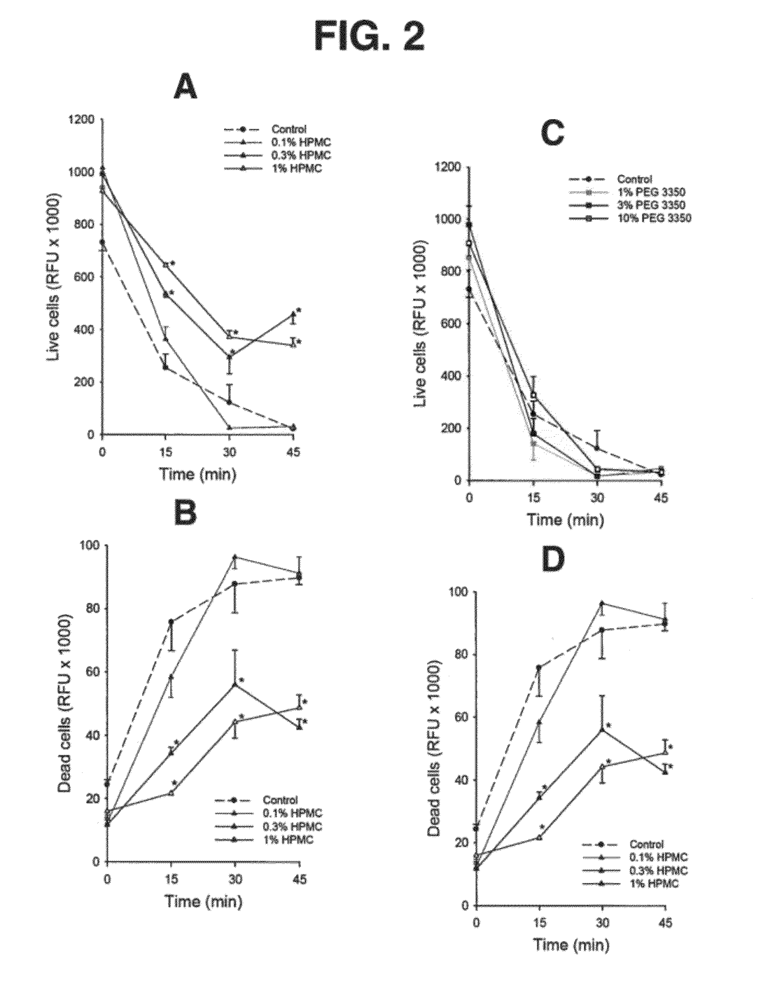

There was a significant increase in calcein fluorescence (indicating increased live cells) after exposure of cells to 0.3% or 1% HPMC 2910 as compared to media control after 15, 30, or 45 minutes of desiccation (FIG. 2). There was significantly less ethidium fluorescence (indicating a decrease in cell death) in cells exposed to 0.3% or 1% HPMC 2910 after 15, 30, or 45 minutes (FIG. 2).

There was no significant difference in calcein or ethidium fluorescence in cells exposed to 0.1% HPMC 2910 or 1%, 3%, or 10% PEG 3350 after 15-45 min desiccation (FIG. 2).

### Summary of Findings

HPMC 2910 (0.3% and 1%) decreased desiccation-induced HCEpiC death at all the time points measured (15, 30 and 45 min). Thus, a composition of the present invention including a non-ionic cellulose derivative, such as HPMC, can provide improved viability to the corneal surface against desiccation.

There was no significant effect of 0.1% HPMC 2910 or 1%, 3% or 10% PEG 3350 on desiccation-induced cell death of HCEpiC.

### Testing 4: Effect of Hyperosmolarity and 10% PEG-3350 on Monolayer Resistance in Riken Transformed Human Corneal Epithelial Cells

### Introduction

In this study, electrical cell-substrate impedance sensing (“ECIS”) was used to effectively monitor changes in Riken transformed human epithelial cells (“RT-HCEpiC”) monolayer resistance, an indicator of barrier function. We recently devised this novel ECIS system to determine the effect of osmolarity using sodium chloride (NaCl) on monolayer resistance of human epithelial conjunctival cells, indicating a change in monolayer permeability. First, we sought to demonstrate that ECIS can be used to monitor changes in cell monolayer resistance induced by NaCl or sucrose hyperosmotic medium. Second, these experiments examined the ability of PEG-3350 to increase monolayer barrier function to hyperosmotic induced monolayer resistance change.

### Method

RT-HCEpiC cells were seeded on ECIS 8-well slide in DMEM/F12 medium containing 15% FBS and HCGS (DMEM/F12 complete HCGS medium) (0.25 ml/well) at a density of 1 or 2×105 per mL and cultured until they reach confluence (−2-3 days after seeding) in an incubator at 37° C., 5% CO₂, and 95% humidity. Culture medium was removed by aspiration and cells were incubated in basal medium containing test ingredient at concentration listed in Table 1. Cells were cultured under these conditions and the change in resistance was monitored by ECIS at 20 minute intervals. One well of cells was tested with basal medium only as a negative control and one was tested with 33 ppm Benzododecinium bromide (BOB) as a positive control per slide for measurement of the resistance change.

TABLE T-4-1
Experimental Design and Schedule Summary: NaCl Hyperosmolarity

| | | | Day 3 |
|---|---|---|---|
| | | | Cells were pretreated in basal |
| | | | DMEM/F12 for 16 hours |
| | | | (0.25 ml) followed by treatment |
| | | | shown below and chamberslide |
| | Slide | | wells were monitored for resistance |
| Group | (well) | Day 1-2 | every 20 min at 3000 kHz for 3 hours. |
| 1 | 1, 3 (1) | Seed RT- | 305 mOsm/kg basal medium |
| | | HCEpiC | DEM/F12 (Control) |
| 2 | 1-4 (5) | (2 × | 33 ppm BOB added to basal medium |
| 3 | 2, 4 (1) | 10⁵/well in | 10% PEG 3350 in basal medium |
| | | 250 μl | DEM/F12 |
| 4 | 1 (2, 6) | DMEM | 460 mOsm/kg medium (NaCl) |
| 5 | 1 (3, 7) | F12 | 535 mOsm/kg medium (NaCl) |
| 6 | 1 (4, 8) | complete | 610 mOsm/kg medium (NaCl) |
| 7 | 2 (2, 6) | and grown | Pretreat 2 hours in 10% PEG, then |
| | | to | 460 mOsm/kg medium (NaCl) |
| 8 | 2 (3, 7) | confluence | Pretreat 2 hours in 10% PEG, then |
| | | | 535 mOsm/kg medium (NaCl) |
| 9 | 2 (4, 8) | | Pretreat 2 hours in 10% PEG, then |
| | | | 610 mOsm/kg medium (NaCl) |

TABLE T-4-2
Experimental Design and Schedule Summary: Sucrose Hyperosmolarity

| | | | Day 3 |
|---|---|---|---|
| | | | Cells were pretreated in basal |
| | | | DMEM/F12 for 16 hours |
| | | | (0.25 ml) followed by treatment |
| | | | shown below and chamberslide |
| | Slide | | wells were monitored for resistance |
| Group | (well) | Day 1-2 | every 20 min at 3000 kHz for 24 hours. |
| 1 | 1, 3 (1) | Seed RT- | Basal Medium Control DEM/F12 |
| 2 | 1-3 (5) | HCEpiC | 25 ppm BOB added to basal medium |
| 3 | 1 (2, 6) | (2 × | 465 mOsm/kg medium (sucrose) |
| 4 | 1 (3, 7) | 10⁵/well in | 525 mOsm/kg medium (sucrose) |
| 5 | 1 (4, 8) | 250 μl | 585 mOsm/kg medium (sucrose) |
| 6 | 2 (2, 6) | DMEM | 2 hours in 10% PEG, then 465 |
| | | F12 | mOsm/kg medium (sucrose) |
| 7 | 2 (3, 7) | complete | 2 hours in 10% PEG, then 525 |
| | | and | mOsm/kg medium (sucrose) |
| 8 | 2 (4, 8) | grown to | 2 hours in 10% PEG, then 585 |
| | | confluence | mOsm/kg medium (sucrose) |
| 9 | 3 (2-4) | | 645 mOsm/kg medium (sucrose) |
| 10 | 3 (6-8) | | 2 hours in 10% PEG, then 645 |
| | | | mOsm/kg medium (sucrose) |

### Data Analysis

Integrated responses of the change in impedance were analyzed by calculating the areas under the curve for each test well over the time course using the trapezoidal rule, which is defined by the equation below:

Σ(Time(Hr)n)−(n−1))×(Impedance(Ohms)n+n−1)/2=Ohms*Hr

Integrated responses were analyzed by a two-way ANOVA followed by the Tukey-Kramer test. Prior to statistical analysis, data were evaluated for normality and variance homogeneity and, if needed, results were subjected to Box-Cox transformations. Any transformation of the data is listed in the figure legend.

### Summary of Findings

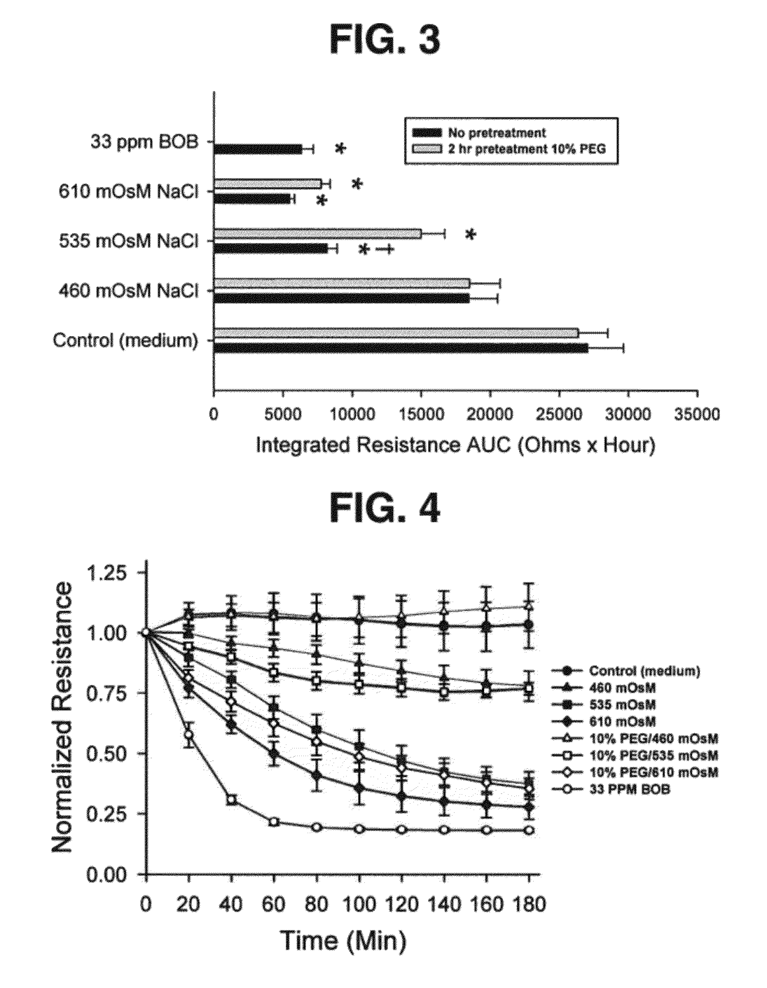

Treatment of cells with 460 mOsm/kg NaCl medium with or without PEG 3350 did not significantly reduce integrated monolayer resistance as measured by ECIS as compared to basal medium control (FIG. 3).

Treatment of cells with 535 mOsm/kg and 610 mOsm/kg NaCl medium with or without PEG 3350 significantly reduced integrated monolayer resistance compared to basal medium control. (FIG. 3).

Treatment of cells with 535 mOsm/kg NaCl medium without PEG 3350 had significantly reduced integrated monolayer resistance as compared to cells treated with 535 mOsm/kg NaCl and pretreated with PEG 3350 (FIG. 3).

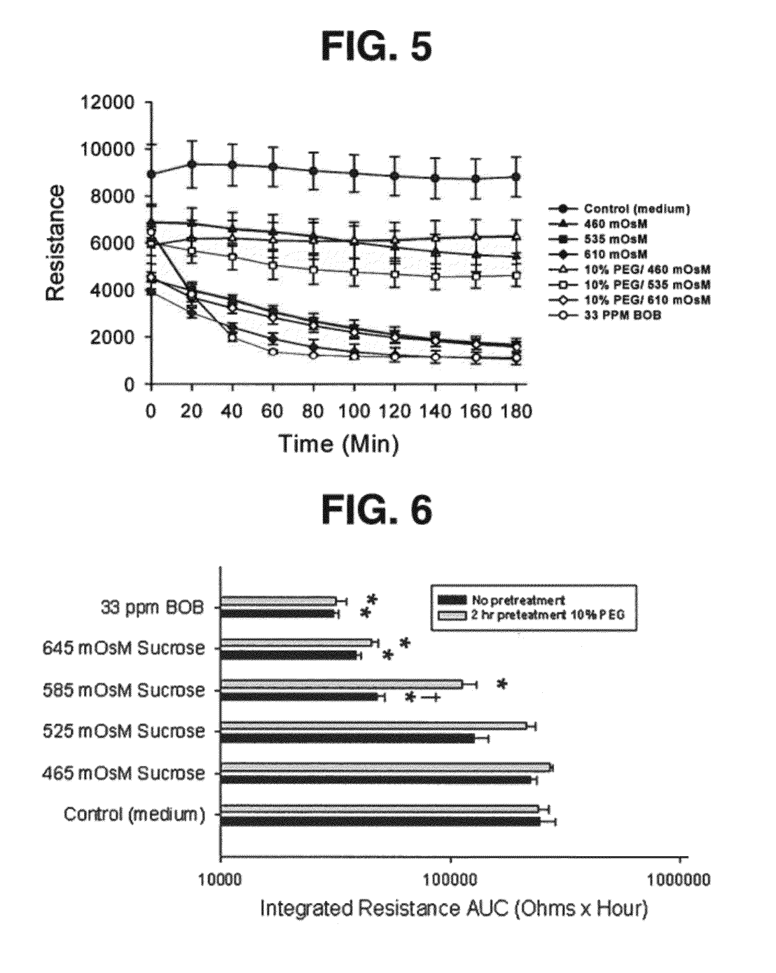

Time course of the normalized and raw monolayer resistance to time 0 after treatments with PEG 3350 and basal and NaCl hyperosmolar medium is shown in FIGS. 4 and 5 respectively.

Treatment of cells with 465 and 525 mOsm/kg sucrose medium with or without PEG 3350 did not significantly reduce integrated monolayer resistance as measured by ECIS as compared to basal medium control (FIG. 6).

Treatment of cells with 585 mOsm/kg and 645 mOsm/kg sucrose medium with or without PEG 3350 significantly reduced integrated monolayer resistance compared to basal medium control. (FIG. 6).

Treatment of cells with 585 mOsm/kg sucrose without PEG 3350 pretreatment had significantly reduced integrated monolayer resistance compared to cells treated with 585 mOsm/kg sucrose and PEG 3350 pretreatment. (FIG. 6).

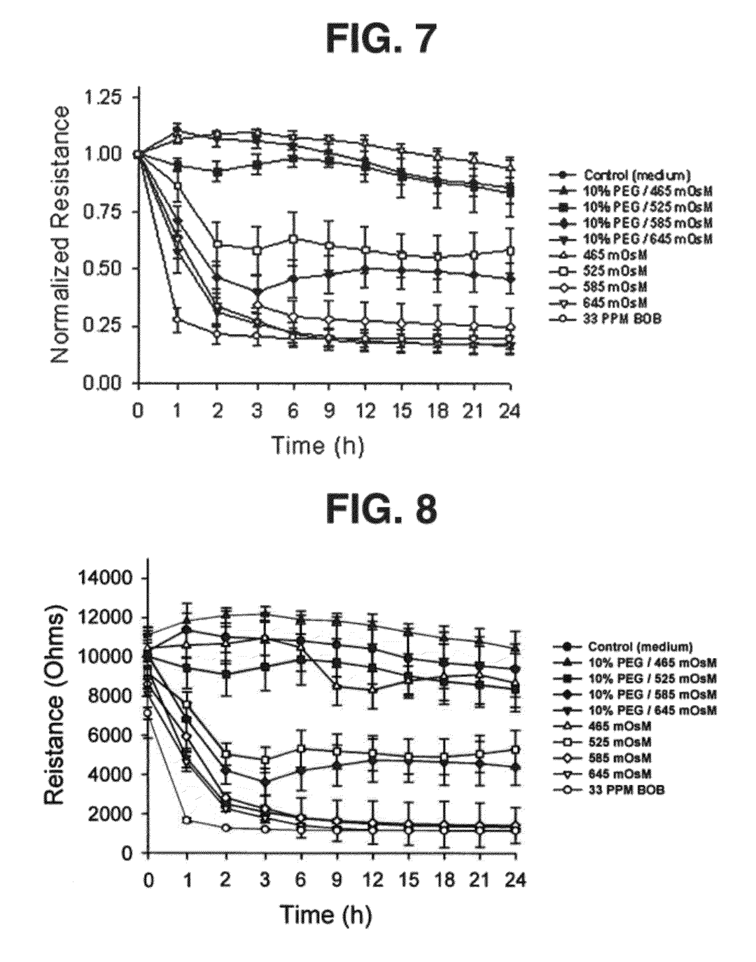

Time course of the normalized and raw monolayer resistance to time 0 after treatments with PEG 3350 and basal and sucrose hyperosmolar medium is shown in FIGS. 7 and 8 respectively.

Testing 5: Effect of Peg-3350 on Mucin (MUC1 and MUC16) mRNA Levels in Riken Human Corneal Epithelial Cells

### Introduction

Dry eye is defined by the DEWS Definition and Classification Subcommittee as a multifactorial disease of the tears and ocular surface that results in symptoms of discomfort, visual disturbance and tear instability with potential damage to the ocular surface, accompanied by increased osmolarity of the tear film and inflammation of the ocular surface.

Ocular surface mucins are crucial to maintain the stability of the tear film, provide lubrication and maintain corneal and conjunctival epithelial cell barrier function. Three membrane associated mucins, MUC1, MUC4 and MUC16 have been shown to be expressed in corneal epithelium, with MUC1 and MUC16 being the more highly expressed (see I. K. Gipson, “Distribution of Mucins at the Ocular Surface,” Exp. Eye Res., Vol. 78, 379-88 (2004)). Several papers have demonstrated that dry eye patients have altered levels of MUC1 and MUC16. A recent study showed that MUC1 was significantly lower in the conjunctival epithelium of patients with aqueous deficient dry eye (R. M. Corrales et al., “Ocular Mucin Gene Expression Levels as Biomarkers for the Diagnosis of Dry Eye Syndrome,” Invest. Ophthaimol. Vis. Sci., Vol. 52, 8263-69 (2011)). Another study has shown that patients with dry eye express less of the MUC1/A splice variant than normal control group (Y. Imbert et at., “MUGC1 Splice Variants in Human Ocular Surface Tissues: Possible Differences Between Dry Eye Patients and Normal Controls,” Exp. Eye Res., Vol. 83, 493-501 (2006)). In Sjogrens syndrome dry eye patients, there was an increase in MUC1 and MUC16 mRNA and protein levels in conjunctival and tear samples (B. Caffery et al., “MUC1 Expression in Sjogren's Syndrome, KCS, and Control Subjects,” Mol. Vis., Vol. 16, 1720-27 (2010); B. Caffery et al., “MUC16 Expression in Sjogren's Syndrome, KCS, and Control Subjects,” Mol. Vis., Vol. 14, 2547-55 (2008)). The goal of this study was to determine the effect of PEG-3350 at varying time points on MUC1 and MUC16 mRNA levels in Riken (SV40) transformed human corneal epithelial cells (“RT-HCEpiC”).

### Method

### Design

Human corneal epithelial cells (“RT-HCEpiC”) were seeded in four 12-well plate in complete culture medium (DMEM/F12+10% fetal bovine serum (“FBS”)). Cells were cultured for 1 week (for approximately 3 days after becoming confluent). Culture medium was replaced with DIMEM/F12+10% charcoal-stripped FBS for 18 h prior to treatment. HCEpiC were then treated with DMEM/F12 basal medium with or without 3%, or with 10% PEG-3350 for 0-24 h; or with 10% PEG-3350 for 2 hours, followed by incubation in DMEM/F12 basal medium for 4-24 h (Table T-5-1). Total RNA was isolated from the cells using an RNeasy Plus mini kit, and then quantified using a Quant-It RNA kit from Invitrogen. An affinity script qPCR cDNA synthesis kit was used to prepare cDNA, QPCR was performed to determine the mRNA expression of MUC1 and MUC16. Glucuronidase beta (GUSB) was used as a housekeeping gene.

TABLE T-5-1

| | | | Day 2 | |
|---|---|---|---|---|
| | | | Cells were incubated with the test | |
| | | | agents in DMEM/F12 basal | |
| Group* | Plate | Day 1 | media for the indicated times. | Day 3 |
| 1 | 1 | Cells were | Control (basal medium), 4 hours | Total RNA |
| 2 | 1 | seeded in | Basal medium + 3% PEG, 4 | was isolated |
| | | four 12-well | hours | from the cells |
| 3 | 1 | plates (1.5 × | Basal medium + 10% PEG, 4 | and stored at |
| | | 10⁵/well in 1 ml | hours | −70° C. for |
| 4 | 1 | medium) | In 10% PEG for 2 hours, then 2 | qPCR |
| | | in complete | hours in basal medium | analysis. |
| 5 | 2 | DMEM/F12. | Control (basal medium), 8 hours | |
| 6 | 2 | | Basal medium + 3% PEG, 8 | |
| | | | hours | |
| 7 | 2 | | Basal medium + 10% PEG, 8 | |
| | | | hours | |
| 8 | 2 | | In 10% PEG for 2 hours, then 6 | |
| | | | hours in basal medium | |
| 9 | 3 | | Control (basal medium), 18 hours | |
| 10 | 3 | | Basal medium + 3% PEG, 18 | |
| | | | hours | |
| 11 | 3 | | Basal medium + 10% PEG, 18 | |
| | | | hours | |
| 12 | 3 | | In 10% PEG for 2 hours, then 16 | |
| | | | hours in basal medium | |
| 13 | 4 | | Control (basal medium), 24 hours | |
| 14 | 4 | | Basal medium + 3% PEG, 24 | |
| | | | hours | |
| 15 | 4 | | Basal medium + 10% PEG, 24 | |
| | | | hours | |
| 16 | 4 | | In 10% PEG for 2 hours, then 22 | |
| | | | hours in basal medium | |

*triplicate wells per group

### Data Analysis

Amplification plots were examined to verify that each consists of a linear baseline region, log phase of amplification, followed by a plateau. To confirm the linearity and efficiency of the reaction and that the efficiency of MUC1 or MUC16 DNA synthesis was similar to GUSB, a correlation plot was generated by subjecting a serial dilution of selected samples to amplification and then plotting the relative amounts against the measured threshold cycle (Ct) values using the Mx3005P software. The R² value was <0.99 for all correlation plots, indicating linearity of the reactions. Efficiency was typically greater than 80% and was equivalent for MUC and GUSB.

To quantify the fold increase in MUC1 and MUC16 mRNA expression over control with the various treatments, the Mx3005P software calculated the relative quantification data where the expression levels of the PEG-3350 samples were compared to the control samples after normalization for the endogenous control GUSB. The data was expressed in fold differences of gene expression compared to control (at 4 hours).

Data were expressed as mean±SEM (standard error of the mean). Statistical analysis was performed using a two-way ANOVA-Tukey Kramer test (Factor 1 was time; Factor 2 was treatment; JMP 8 software). p<0.05 was considered statistically significant. Data were analyzed either directly or after Box-Cox transformations.

### Results

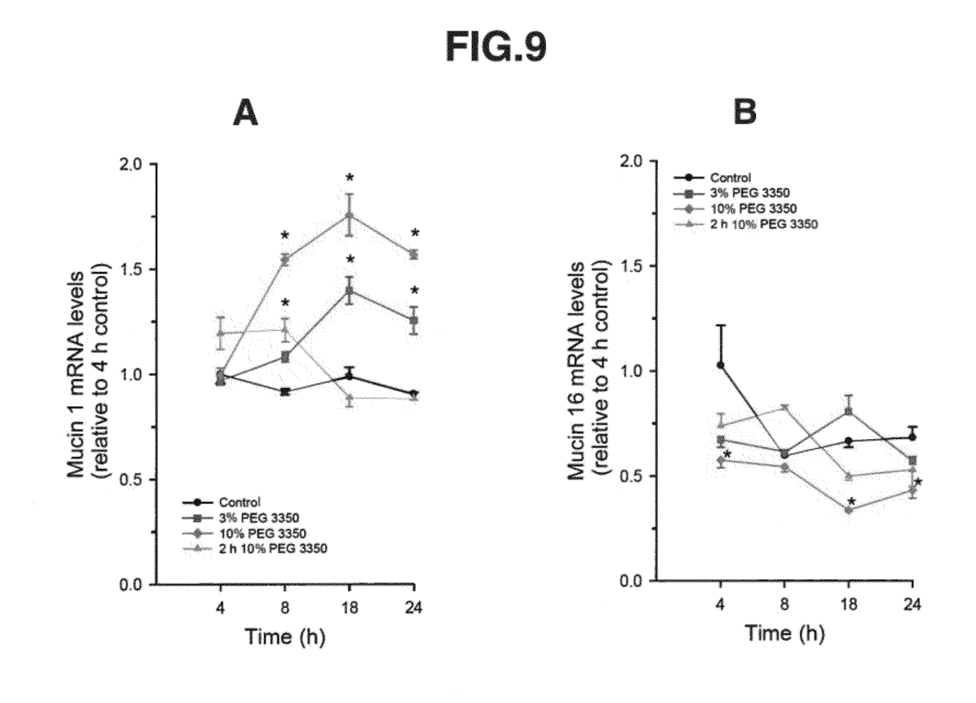

There was a significant increase in MUC1 mRNA levels after HCEpiC were treated with 10% PEG-3350 for 8, 18, or 24 hours; 3% PEG-3350 for 18 or 24 hours; or 2 hours in 10% PEG followed by 6 hours in control basal medium (FIG. 9A).

There was a significant decrease in MUC16 mRNA levels in HCEpiC exposed to 10% PEG 3350 for 4, 18, or 24 hours (FIG. 9B).

### Summary of Findings

PEG-3350 at 10% increased MUC1 mRNA levels at 8, 18, or 24 hours. PEG-3350 at 3% elevated MUC1 mRNA levels at 18, or 24 hours. In addition, a 2-hour treatment with PEG-3350 followed by 6-hour incubation in control basal medium resulted in the significant increase in MUC1 mRNA. Thus, a composition of the present invention including a polyethylene glycol, such as PEG-3350, can stimulate the production of mucin in the eye.

There was a significant decrease in MUC16 mRNA levels with 10% PEG-3350 at 4, 18, or 24 hours.

### Testing 6: Effect of PEG-3350 (10%) on Activation of Cell Signaling Pathways in Riken Transformed Human Corneal Epithelial Cells

### Introduction

Successful wound healing involves a number of cellular processes, particularly cell migration and proliferation (see; e.g., F. S. X. Yu et al., “Growth Factor and Corneal Epithelial Wound Healing,” Brain Res. Bull., Vol. 81, No. 2-3, 229-35 (2010)). These processes are initiated and coordinated by growth factors generated in large part by the cornea in response to wounding. The epidermal growth factor receptor (“EGFR”) is a transmembrane tyrosine kinase receptor activated upon corneal wounding and is necessary for initiation of migration and healing (see J. S. Lozano et al., “Activation of the Epidermal Growth Factor Receptor by Hydrogels in Artificial Tears,” Exp. Eye Res., Vol. 86, 500-05 (2008)). EGFR is active in a phosphorylated state, providing binding sites for numerous signaling molecules, including extracellular signal-regulated kinase 1 and 2 (“ERK1/2”). ERK1/2 has been shown to contribute to corneal wound healing by promoting cell proliferation and migration (see F. S. X. Yu et al., “ERK1/2 Mediate Wounding and G-Protein Coupled Receptor Ligands Induced EGFR Activation via Regulating ADAM 17 and HB-EGF Shedding,” Invest. Ophthahnol. Vis. Sci., Vol. 50, No. 1, 132-39 (2009)).

In this study, we sought to elucidate the molecular mechanisms behind the observed positive effect of 10% PEG-3350 on corneal re-epithelization. In particular, the goal of this study is to assess the phosphorylation states of EGFR, ERK, Akt, and PI3K following exposure to 10% PEG-3350 over the course of 16 hours.

### Methods

### Western Blotting

RT-HCEpiC cell suspensions (2.5×105 cells/ml) were prepared in complete medium with 10% Fetal Bovine Serum (“FBS”) and added to each well of a 6-well plate, 2.5 mL suspension per well. When cells reached confluence, they were serum starved overnight in basal DMEM/F12. Treatments were delivered in basal medium; treatments include control, 10 ng/mL HB-EGF (Heparin-bi-binding EGF-like growth factor) for 10 minutes; or 10% PEG-3350 for 10, 15, 20, 30 minutes, 1, 2, 4, 6, or 16 hours. Cell lysates were assayed for protein concentration and evaluated for Akt, ERK, EGFR, and PI3K activation by Western blot.

Culture medium was aspirated from each well and cells were washed with cold, non-sterile PBS twice. Cells were incubated with 1×SDS lysis buffer and then scraped to the bottom of each well and transferred to microfuge tubes. Cell lysates were sonicated to homogenize the sample followed by centrifugation at 10 minutes×13,000 RPM. Supernatants containing cell lysates were transferred to fresh microfuge tubes and stored at −70° C. Cell lysates were assayed for protein concentration and analyzed by western blot. After probing for phosphorylated proteins, blots were stripped and re-probed for corresponding total protein. Western blots were imaged via chemiluminescent detection with the Bio Rad Versa Doc 4000 MP imager.

TABLE T-6-1
Experimental Design and Schedule Summary

| | | Day 2 | |
|---|---|---|---|
| | | Cells were serum | |
| | | starved in DMEM/F12 | |
| | | without supplements | |
| | | overnight. Treatment groups | |
| Group* | Day 1 | applied accordingly. | Day 3 |
| 1 | Cells were | Control | Cell lysates |
| 2 | seeded to 6 | 10 minutes, 10 ng/mL HB-EGF | were |
| 3 | well plates | 10 minutes, 10% PEG-3350 | collected for |
| 4 | in | 15 minutes, 10% PEG-3350 | Western |
| 5 | DMEM/F12 + | 20 minutes, 10% PEG-3350 | blot. |
| 6 | 10% FBS. | 30 minutes, 10% PEG-3350 | |
| 7 | | 1 hour, 10% PEG-3350 | |
| 8 | | 2 hours, 10% PEG-3350 | |
| 9 | | 4 hours, 10% PEG-3350 | |
| 10 | | 6 hours, 10% PEG-3350 | |
| 11 | | 16 hours in Control medium | |
| 12 | | 16 hours, 10% PEG-3350 | |

### Data Analysis

Protein Measurement: Absorbance at 570 nm (OD) was used to determine protein concentration in the cell lysates based upon a standard curve created using albumin. Data was analyzed using linear regression following LP06017.

Western Blot: Analysis of Western blot band density in captured and stored digital images was done using the Quantity One software on the VersaDod MP 4000. Density of bands was quantified. Results are reported as values ratios of phosphorylated protein to total protein.

### Results

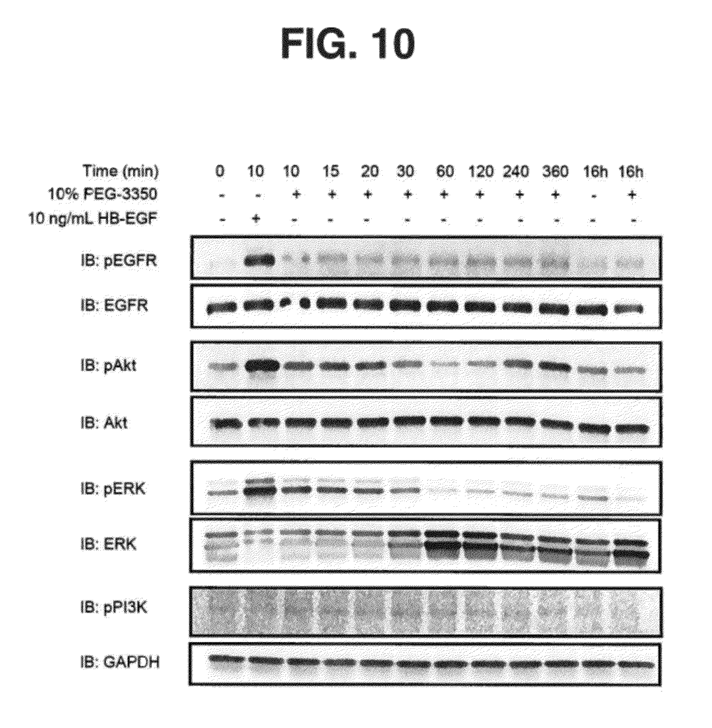

The treatment with 10 ng/mL HB-EGF (a positive control) for 10 minutes substantially increased phosphorylation of EGFR, Akt and ERK, but no effect was observed with respect to pPI3K (FIG. 10).

10% PEG-3350 increased phosphorylation of EGFR as early as 10 minutes and was sustained out to 6 hours, and to a lesser extent, after 16 hours (FIG. 10).

Phosphorylation of Akt was observed and sustained from 10 to 20 minutes after incubation with 10% PEG-3350. There was a slight decrease in phosphorylation at and 60 minutes, followed by an increase at 120 and 240 minutes with peak phosphorylation seen after 6 hours (FIG. 10).

10% PEG-3350 activated ERK as shown by phosphorylation at 10, 15, or 20 minutes, and with a slight decline at 30 minutes (FIG. 10). At 60 minutes, ERK phosphorylation was less than in control cells. There was a slight increase toward control levels of phosphorylation at 6 hours. However, following 16 hours with 10% PEG-3350 ERK phosphorylation had decreased to sub-control levels.

No observable change in phosphorylation of PI3K was detected at any of the time points treated with 10% PEG-3350 (FIG. 10).

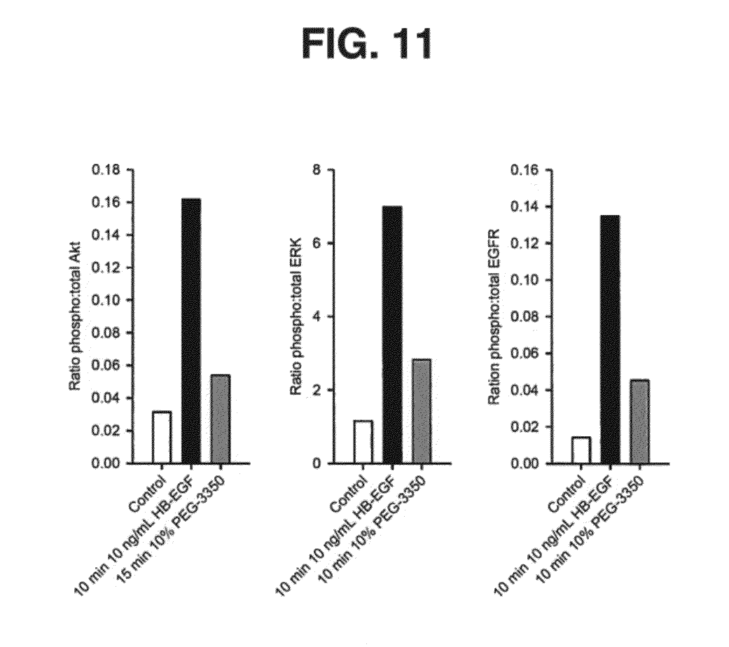

FIG. 11 shows graphs representative of peak protein phosphorylation time-points for respective proteins. Ratio of phosphorylated protein to total, non-phosphorylated was quantified by densitometry.

### Summary of Findings

10% PEG-3350 activated phosphorylation of EGFR, Akt, and ERK at various points along the duration of 16 hours.

As an activator of certain proteins involved in wound healing signaling, 10% PEG-3350 was far less potent than 10 ng/mL HB-EGF, 10 minutes.

Thus, a composition of the present invention including a polyethylene glycol, such as PEG-3350, can activate cell signaling pathway involving EGFR, Akt, or ERK, to promote healing of an impaired corneal epithelial layer.

### Testing 7: Effect of Hyperosmolarty and 3% or 10% Polyethylene Glycol 33500N Distribution of ZO-1 and Actin in Riken Transformed Human Corneal Epithelial Cells

### Introduction

Epithelial barrier function is maintained by tight junctions. Tight junctions are composed of a complex of proteins which form a tight contact between the plasma membrane of adjacent cells (a. Nusrat et al., “Molecular Physiology and Pathophysiology of Tight Junctions. IV. Regulation of Tight Junctions by Extracellular Stimuli: Nutrients, Cytokines, and Immune Cells,” Am. J. Physiol. Gatroinstest. Liver Physiol., Vol. 279, G851-857 (2000)). Tethered to the tight junctions and crucial for their integrity is the actin cytoskeleton, which is organized as a peri-junctional actin ring in corneal epithelial cells (S. P. Srinivas et al., “Histamine-Induced Phosphorylation of the Regulatory Light Chain of Myosin II Disrupts the Barrier Integrity of Corneal Endothelial cells,” Invest. Ophthalmol. Vis. Sci., Vol. 47, 4011-18 (2006)). Loss of epithelial barrier function occurs due to increased contractility and disruption of actin and breakdown of tight junction proteins such as occludin, ZO-1 and ZO-2 (K. Araki-Sasaki et al., “An SV40-Immortalized Human Corneal Epithelial Cell Line and its Characterization,” Invest Ophthalmol. Vis. Sci., Vol. 36, 614-21 (1995)). In this study, we studied the effects of 10% PEG-3350 pretreatment on the changes induced by sucrose hyperosmolarity in the distribution of ZO-1 and actin in HCEpiC 3350, using confocal microscopy.

### Methods

### Cell Culture

RT-HCEpiC cells were seeded on 4-well chamberslide in DMEM/F2 medium containing 15% fetal bovine serum (“FBS”) and Human Corneal Growth Supplement (“HCGS”) (0.5 ml/well) at a density of 5×104 per mL and cultured until they reach confluence (−2-3 days after seeding) in an incubator at 37° C., 5% CO₂, and 95% humidity. Confluent cells were cultured in 15% FBS HCGS medium for 2-3 more days to ensure formation of tight junctions. Culture medium was removed by aspiration and cells were incubated in DMEM/F12 serum free medium for 16 h prior to incubation in the test treatments. Culture medium was removed by aspiration and cells were incubated in basal medium containing PEG 3350 or hyperosmolar sucrose medium at the concentration as described in Table T-7-1 and T-7-2.

TABLE T-7-1
Experimental Design for Immunocytochemistry

| | | Day 5-6 |
|---|---|---|
| | | Cells was exposed to |
| | | pretreatment in basal DMEM/F12 |
| | | medium, or 3% or 10% PEG-3350 |
| | | in basal medium, for 2 hours |
| | | prior to treatment with hyperosmolar |
| Group* | Day 1-6 | sucrose medium for 2 hours. |
| 1 | Seed RT-HCEpiC (5 × | Medium Control (Basal DMEM/F12) |
| 2 | 10⁴/well in 0.5 ml | 465 mOsm/kg medium (Sucrose) |
| 3 | DMEM F12 Complete | 525 mOsm/kg medium (Sucrose) |
| 4 | and grown to | 585 mOsm/kg medium (Sucrose) |
| 5 | confluence. Confluent | 2 hours in 10% PEG, then 465 |
| | cells were cultured in | mOsm/kg medium (Sucrose) |
| 6 | 15% FBS HCGS | 2 hours in 10% PEG, then 525 |
| | medium for 48-72 | mOsm/kg medium (Sucrose) |
| 7 | hours. | 2 hours in 10% PEG, then 585 |
| | | mOsm/kg medium (Sucrose) |

*3 images per group

TABLE T-7-2
Experimental Design for Immunocytochemistry

| | | Day 5-6 |
|---|---|---|
| | | Cells was exposed to |
| | | pretreatment in basal DMEM/F12 |
| | | medium, or 10% PEG-3350 |
| | | in basal medium for 2 hours |
| | | prior to treatment with hyperosmolar |
| Group* | Day 1-2 | sucrose medium for 2 hours. |
| 1 | Seed RT-HCEpiC (5 × | Medium Control (Basal DMEM/F12) |
| 2 | 10⁴/well in 0.5 ml | 525 mOsm/kg medium (Sucrose) |
| 3 | DMEM F12 Complete | 585 mOsm/kg medium (Sucrose) |
| 4 | and grown to | 2 hours in 10% PEG, then 525 |
| | confluence. | mOsm/kg medium (Sucrose) |
| 5 | | 2 hours in 10% PEG, then 585 |
| | | mOsm/kg medium (Sucrose) |

*4 images per group

### Immunocytochemistry for ZO-1 and Actin:

Treatment solutions were removed by aspiration and cells were washed in phosphate buffered saline (PBS) with 0.5 mM magnesium chloride and 1 mM calcium chloride (PBS-CM). Cells were fixed for 10 minutes in 3.7% paraformaldehyde, followed by 3 washes in PBS and then a 10 minute neutralization in 20 mM glycine. Cells were washed 3× in PBS and then permeabilized in PBS/0.1% TritonX-100 (TX-100) for 10 minutes prior to blocking with 1% BSA with 10% goat serum in PBS-CM for 30 minutes. After blocking, cells were incubated in 500 μl PBS with ZO-1 antibody at 1:500 16 hours at 4° C. on a rocker. Cells will then be washed 3×10 minutes in PBS on a rocker. Cells were incubated in 500 μl PBS/1% BSA+Alexa-fluor rabbit 488 at 1:2000+10 μl Alexa-fluor 568 phalloidin (a small molecule which specifically binds to actin-filaments) for 1 hour. Cells were washed 3×10 minutes in PBS-Triton X-100. The walls and gasket of the chamber slide were removed and I drop of vectashield with propidium iodide was added to each chamber well. A glass coverslip was placed on top and the edges sealed with nail polish. The cells were viewed using the confocal microscope at either 10× or 20× magnification.

### Results

TABLE T-7-3
ZO-1 and Actin Distribution

| | Osmolality | | |
|---|---|---|---|
| PEG Pretreatment | (mOsm/kg) | ZO-1¹ | Actin¹ |
| None | 305 (basal) | ++++² | ++++ |
| None | 525 | +++ | +++ |
| None | 585 | ++ | ++ |
| 10% | 305 (basal) | ++++ | ++++ |
| 10% | 525 | ++++ | ++++ |
| 10% | 585 | +++ | +++ |

¹From FIGS. 15-17
²All data as compared to no PEG-3350 pretreatment followed by 2 hours in basal medium. ++++ represents no change in the distribution of ZO-1 or actin proteins.

### Summary of Findings

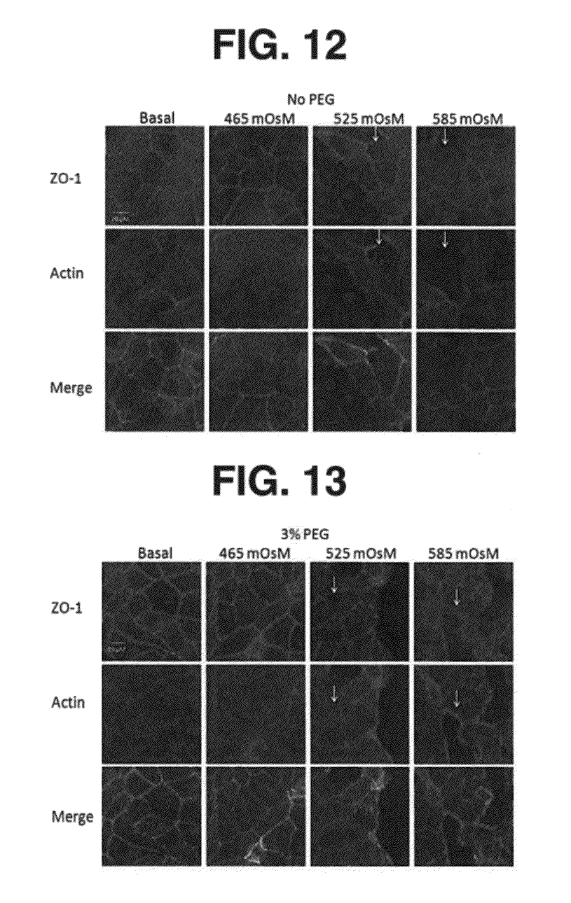

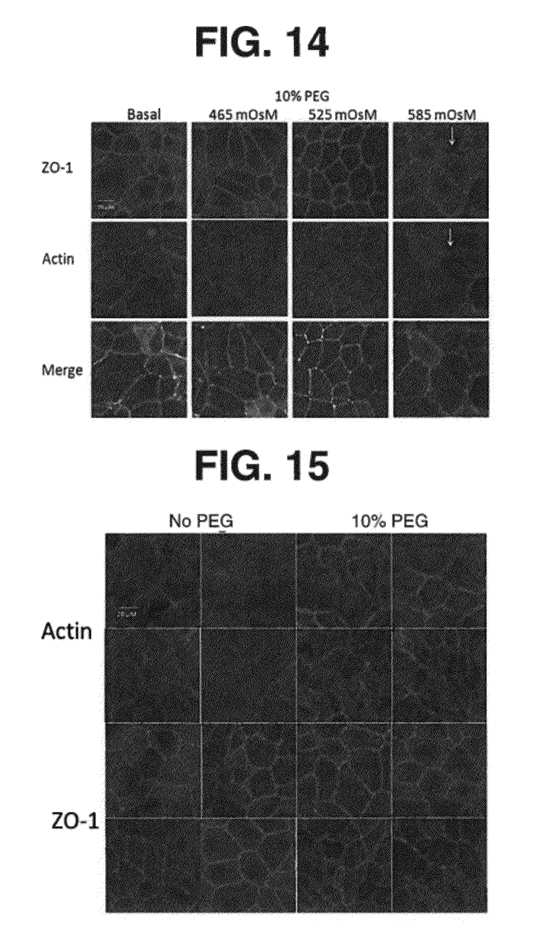

The distribution of ZO-1 and actin proteins after no PEG-3350 pretreatment or 3% or 10% PEG-3350 followed by increasing hyperosmotic medium are shown in FIGS. 12-14. For the cells not receiving PEG pretreatment, changes to ZO-1 and actin distribution were observed with the 525 and 585 mOsm/kg sucrose treated cells but not with 465 mOsm/kg sucrose concentration as compared to cells in basal medium only.

For cells pretreated with 3% PEG-3350, changes to ZO-1 and actin distribution were observed with both the 525 and 585 mOsm/kg sucrose treated cells but not with the 465 mOsm/kg concentration as compared to cells pretreated with 3% PEG-3350 followed by incubation in basal medium.

For cells pretreated with 10% PEG-3350, changes to ZO-1 and actin distribution were observed only with the 585 mOsm/kg sucrose treated cells. The 465 and 525 mOsm/kg sucrose treatments had no effect on ZO-1 and actin distribution after 10% PEG-3350 pretreatment.

To distinguish subjective differences between the 525 and 585 mOsm/kg sucrose treatments a more rigid assessment was made in which 4 random images from cells with or without 10% PEG pretreatment followed by treatment in these two hyperosmotic media was performed and the results are summarized in Table T-7-3 and shown in FIGS. 15-17.

FIG. 15 shows a comparison of cells with or without 10% PEG pretreatment followed by treatment in basal medium. No differences in ZO-1 or actin distribution were observed between these groups and both were assessed 4+ (Table T-7-3) indicating no changes for both ZO-1 and actin distribution.

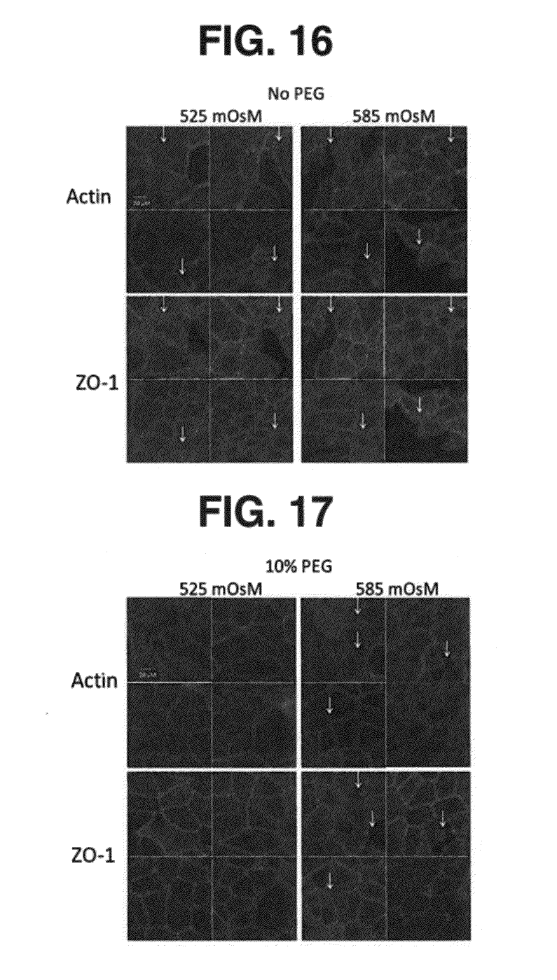

FIG. 16 shows a comparison of cells without PEG pretreatment followed by treatment in either 525 or 585 mOsm/kg sucrose. The cells treated with 525 mOsm/kg sucrose were assessed at 3+ for both ZO-1 and actin distribution indicating some disruption was observed. The cells treated with 585 mOsm/kg sucrose were assessed at 2+ for both ZO-1 and actin distribution indicating significant disruption of the tight junctions and a considerable number of large gaps within the cell monolayer was observed.

FIG. 17 shows a comparison of cells with 10% PEG pretreatment followed by treatment in either 525 or 585 mOsm/kg sucrose. The cells treated with 525 mOsm/kg sucrose were assessed at 4+ for both ZO-1 and actin distribution indicating no changes were observed. The cells treated with 585 mOsm/kg sucrose were assessed at 3+ for both ZO-1 and actin distribution indicating some disruption was observed.

### Testing 8: Effect of Hyperosmolarity and 3% PEG-3350 on Monolayer Resistance in Riken Transformed Human Corneal Epithelial Cells

### Introduction

In this study, electrical cell-substrate impedance sensing (ECIS) was used to effectively monitor changes in Riken transformed human epithelial cells (RT-HCEpiC) monolayer resistance, an indicator of barrier function. We used a novel ECIS system to determine the effect of osmolarity using sodium chloride (NaCl) on monolayer resistance of human epithelial conjunctival cells, indicating a change in monolayer permeability. See Testing 4, disclosed hereinabove.

### Methods

RT-HCEpiC cells were seeded on ECIS 8-well slide in DMEM/F12 medium containing 15% FBS and HCGS (DMEM/F12 complete HCGS medium) (0.25 ml/well) at a density of 1 or 2×105 per mL and cultured until they reach confluence (˜2-3 days after seeding) in an incubator at 37° C., 5% CO₂, and 95% humidity. Culture medium was removed by aspiration and cells were incubated in basal medium containing test ingredient at concentration listed in Table T-8-1. Cells were cultured under these conditions and the change in resistance was monitored by ECIS at 20 minute intervals. One well of cells was tested with basal medium only as a negative control and one was tested with 33 ppm Benzododecinium bromide (BOB) as a positive control per slide for measurement of the resistance change. Only sucrose hyperosmolarity was tested in these experiments as NaCl can interfere with the ECIS measurements.

TABLE T-8-1
Experimental Design and Schedule Summary

| | | | Day 3 |
|---|---|---|---|
| | | | Cells were pretreated in basal |
| | | | DMEM/F12 for 16 h (0.25 ml) followed |
| | | | by treatment below and chamberslide |
| | Slide | | wells were monitored for resistance |
| Group | (well) | Day 1-2 | every 20 min at 3000 kHz for 24 hours. |
| 1 | 1-3 (1) | Seed RT- | Basal Medium Control (DMEM/F12) |
| 2 | 1-3 (5) | HCEpiC (5 × | 25 ppm BOB in Basal medium Control |
| 3 | 1 (2, 6) | 10⁴/well in | 465 mOsm/kg medium (sucrose) |
| 4 | 1 (3, 7) | 250 μl | 525 mOsm/kg medium (sucrose) |
| 5 | 1 (4, 8) | DMEM F12 | 585 mOsm/kg medium (sucrose) |
| 6 | 2 (2, 6) | Complete | 2 hours in 10% PEG, then 465 |
| | | and grown to | mOsm/kg medium (sucrose) |
| 7 | 2 (3, 7) | confluence | 2 hours in 10% PEG, then 525 |
| | | | mOsm/kg medium (sucrose) |
| 8 | 2 (4, 8) | | 2 hours in 10% PEG, then 585 |
| | | | mOsm/kg medium (sucrose) |

### Data Analysis

Integrated responses of the change in impedance were analyzed by calculating the areas under the curve for each test well over the time course using the trapezoidal rule, which is defined by the equation below:

Σ(Time(Hr)n)−(n−1))×(Impedance(Ohms)n+n−1)/2=Ohms*Hr

Integrated responses were analyzed by a two-way ANOVA followed by the Tukey-Kramer test. Prior to statistical analysis, data were evaluated for normality and variance homogeneity and, if needed, results were subjected to Box-Cox transformations. Any transformation of the data is listed in the figure legend.

### Summary of Findings.

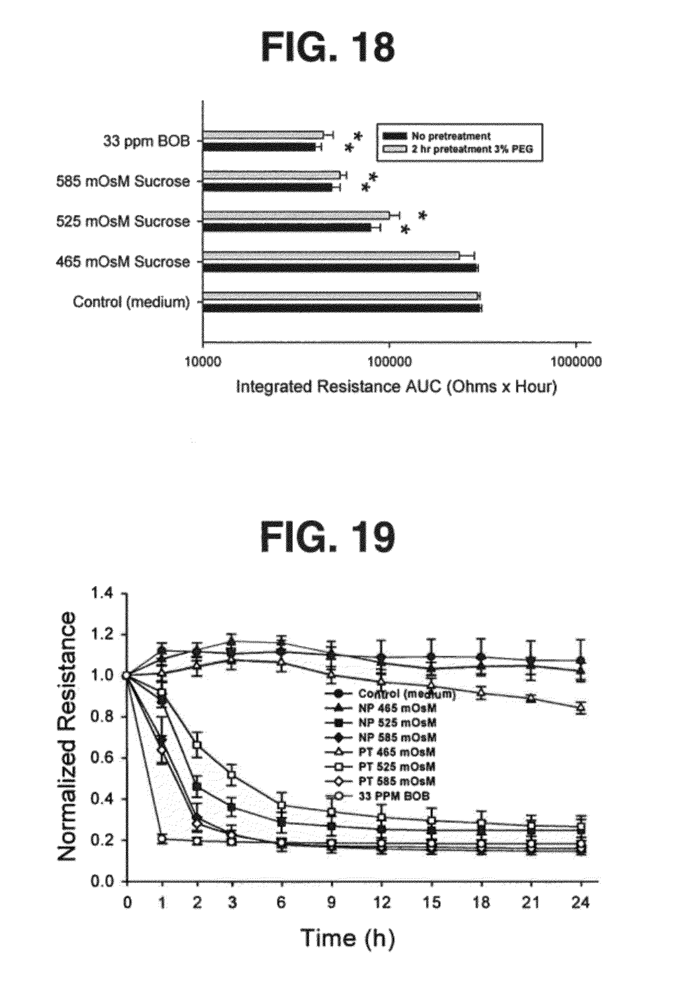

Treatment of cells with 465 mOsm/kg sucrose medium with or without 3% PEG-3350 pretreatment did not significantly reduce integrated monolayer resistance as compared to basal medium control as measured by ECIS as (FIG. 18).

Treatment of cells with 525 mOsm/kg and 585 mOsm/kg sucrose medium with or without 3% PEG-3350 pretreatment significantly reduced integrated monolayer resistance as compared to basal medium control. (FIG. 18).

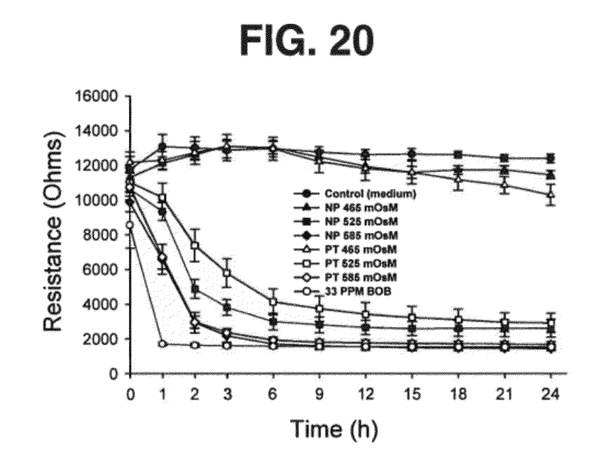

Time course of the normalized and raw monolayer resistance to time 24 hours after pretreatment with PEG-3350 and basal or hyperosmotic sucrose medium are shown in FIGS. 19-20, respectively.

In summary, the results of the studies disclosed herein, taken together, show that a composition of the present invention that comprises a polyethylene glycol such as PEG-3350 or a similar PEG, and a non-ionic cellulose derivative such as HPMC or a similar cellulose derivative can: (1) treat, control, ameliorate, or reverse conditions of dry eye; (2) promote corneal re-epithelization after having been wounded; (3) provide protection to the ocular surface against desiccation-induced cell death; (4) support the integrity of the corneal surface exposed to hyperosmolar insults; (5) promote the production of mucin from the eye leading to improved lubrication of the corneal surface; and (6) promote the activation of cell signaling pathways involving EGFR, ERK, and Akt in the healing process of ocular wounds. All of these findings show that compositions within the scope of the present invention can be effective in treating, controlling, ameliorating, or reversing conditions, symptoms, impairments, or injuries caused by dry eye.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. An ophthalmic composition comprising: (a) a polyethylene glycol having a molecular weigh in the range from about 1,000 to about 10,000 Da at a concentration from about 5 to about 15 percent by weight of the total composition; and (b) a non-ionic water-soluble cellulose derivative having a molecular weight in the range from about 50,000 to about 120,000 Da at a concentration from about 0.1 to about 5 percent by weight of the total composition.

2. The pharmaceutical formulation of claim 1, wherein the polyethylene glycol has a molecular weigh in the range from about 2,000 to about 8,000 Da, and the non-ionic water-soluble cellulose derivative has a molecular weight in the range from about 60,000 to about 100,000 Da.

3. The pharmaceutical formulation of claim 2, wherein the polyethylene glycol is selected from the group consisting of PEG-2000, PEG-3350, PEG-4000, PEG-6000, PEG-8000, and mixtures thereof; and the cellulose derivative is selected from the group consisting of hydroxypropylmethyl cellulose, hydroxypropyl cellulose, hydroxyethyl cellulose, methyl cellulose, and mixtures thereof.

4. The pharmaceutical formulation of claim 3; wherein the concentration of the polyethylene glycol is in the range from about 7 to 12 percent by weight of the total composition, and the concentration of the cellulose derivative is in the range from about 0.3 to 2 percent by weight of the total composition.

5. The pharmaceutical formulation of claim 4; wherein the polyethylene glycol is PEG-3350 or PEG-4000, and the cellulose derivative is hydroxypropylmethyl cellulose.

6. The pharmaceutical formulation of claim 4, further comprising an ophthalmically acceptable therapeutic agent.

7. The pharmaceutical formulation of claim 6; wherein the ophthalmically acceptable therapeutic agent is a compound having Formula II, V, or VI.

8. An ophthalmic composition consisting essentially of: a polyethylene glycol having molecular weight in the range of 2,000 to 10,000; a non-ionic, water-soluble cellulose derivative selected from hydroxypropylmethyl cellulose, hydroxyethyl cellulose, hydroxypropyl cellulose, and methyl cellulose; a non-ionic surfactant selected from polysorbate 20, 60, and 80; boric acid; a polyol selected from the group consisting of glycerin, propylene glycol, and mixtures thereof; at least a phosphate salt; an anti-oxidant; a chelating agent; a preservative; and water; wherein the composition has an osmolality in the range of 260-350 mOsm/kg, a pH in the range of 6.5-8, and a viscosity in the range of 2-500 centipoises.

9. The ophthalmic composition of claim 8; wherein the polyethylene glycol is present at a concentration of 5-15 percent by weight of the total composition; the non-ionic, water-soluble cellulose derivative is present at a concentration of 0.1-2 percent by weight of the total composition; and the non-ionic surfactant is present at a concentration of 0.2-2 percent by weight of the total composition; and the polyol is present at a concentration of 0.01-2 percent by weight of the total composition.

10. A method for treating, controlling, ameliorating, or reversing a condition of dry eye, the method comprising administering into an affected eye a composition that comprises: (a) a polyethylene glycol having a molecular weigh in the range from about 1,000 to about 10,000 Da at a concentration from about 5 to about 15 percent by weight of the total composition; and (b) a non-ionic water-soluble cellulose derivative having a molecular weight in the range from about 50,000 to about 120,000 Da at a concentration from about 0.1 to about 5 percent by weight of the total composition, in an amount and at a frequency effective to treat, control, ameliorate, or reverse said condition.

11. The method of claim 10; wherein said condition is selected from the group consisting of discomfort in the eye, feeling of dryness, grittiness, stinging, irritation in the eye, and deficiency in production of a material of the tear film.

12. The method of claim 10; wherein the polyethylene glycol has a molecular weigh in the range from about 2,000 to about 8,000 Da, and the non-ionic water-soluble cellulose derivative has a molecular weight in the range from about 60,000 to about 100,000 Da.

13. The method of claim 12; wherein the polyethylene glycol is selected from the group consisting of PEG-2000, PEG-3350, PEG-4000, PEG-6000, PEG-800, and mixtures thereof; and the cellulose derivative is selected from the group consisting of hydroxypropylmethyl cellulose, hydroxypropyl cellulose, hydroxyethyl cellulose, methyl cellulose, and mixtures thereof.

14. The method of claim 13; wherein the concentration of the polyethylene glycol is in the range from about 7 to 12 percent by weight of the total composition, and the concentration of the cellulose derivative is in the range from about 0.3 to 2 percent by weight of the total composition.

15. The method of claim 14; wherein the polyethylene glycol is PEG-3350 or PEG-4000, and the cellulose derivative is hydroxypropylmethyl cellulose.

16. The method of claim 14; wherein the composition further comprises an ophthalmically acceptable therapeutic agent.

17. The method of claim 16; wherein the ophthalmically acceptable therapeutic agent is a compound having Formula II, V, or VI.

18. A method for treating, controlling, ameliorating, or reversing a condition of dry eye, the method comprising administering into an affected eye a composition that consists essentially of: a polyethylene glycol having molecular weight in the range of 2,000 to 10,000; a non-ionic, water-soluble cellulose derivative selected from hydroxypropylmethyl cellulose, hydroxyethyl cellulose, hydroxypropyl cellulose, and methyl cellulose; a non-ionic surfactant selected from polysorbate 20, 60, and 80; boric acid; a polyol selected from the group consisting of glycerin, propylene glycol, and mixtures thereof; at least a phosphate salt; an anti-oxidant; a chelating agent; a preservative; and water; wherein the composition has an osmolality in the range of 260-350 mOsm/kg, a pH in the range of 6.5-8, and a viscosity in the range of 2-500 centipoises, in an amount and at a frequency effective to treat, control, ameliorate, or reverse said condition.

19. The method of claim 18; wherein the polyethylene glycol is present at a concentration of 5-15 percent by weight of the total composition; the non-ionic, water-soluble cellulose derivative is present at a concentration of 0.1-2 percent by weight of the total composition; and the non-ionic surfactant is present at a concentration of 0.2-2 percent by weight of the total composition; and the polyol is present at a concentration of 0.01-2 percent by weight of the total composition.

20. The method of claim 19; wherein said condition is selected from the group consisting of discomfort in the eye, feeling of dryness, grittiness, stinging, irritation in the eye, and deficiency in production of a material of the tear film.

21. A method for (1) treating, controlling, ameliorating, or reversing a condition of dry eye; (2) promoting corneal re-epithelization after being wounded; (3) providing protection to an ocular surface against desiccation-induced cell death; (4) supporting the integrity of a corneal surface exposed to hyperosmolar insults; (5) promoting production of mucin from an eye leading to improved lubrication of the corneal surface; and (6) promoting activation of cell signaling pathways involving EGFR, ERK, and Akt in a healing process of ocular wounds, the method comprising administering into an affected eye a composition that comprises: (a) a polyethylene glycol having a molecular weigh in the range from about 1,000 to about 10,000 Da at a concentration from about 5 to about percent by weight of the total composition; and (b) a non-ionic water-soluble cellulose derivative having a molecular weight in the range from about 50,000 to about 120,000 Da at a concentration from about 0.1 to about 5 percent by weight of the total composition, in an amount and at a frequency effective for said treating, controlling, ameliorating, reversing, promoting, or supporting.

22. The method of claim 21; wherein the polyethylene glycol has a molecular weigh in the range from about 2,000 to about 8,000 Da, and the non-ionic water-soluble cellulose derivative has a molecular weight in the range from about 60,000 to about 100,000 Da; and wherein the composition further comprises a non-ionic surfactant and a polyol.

23. The method of claim 22; wherein the polyethylene glycol is present at a concentration of 5-15 percent by weight of the total composition; the non-ionic, water-soluble cellulose derivative is present at a concentration of 0.1-2 percent by weight of the total composition; the non-ionic surfactant is present at a concentration of 0.2-2 percent by weight of the total composition; and the polyol is present at a concentration of 0.01-2 percent by weight of the total composition.
